(19) United States
(12) Patent Application Publication
(10) Pub. No.: US 2024/0271105 A1
(43) Pub. Date: Aug. 15, 2024

(54) MULTI-TISSUE ORGANOID PRODUCTS AND METHODS

(71) Applicant: REGENTS OF THE UNIVERSITY OF MINNESOTA, Minneapolis, MN (US)

(72) Inventors: Timothy D. O'Brien, Cokato, MN (US); Beth Lindborg, St. Paul, MN (US); Amanda Vegoe, Apple Valley, MN (US); Yi Wen Chai, Minneapolis, MN (US); Manci Li, St. Paul, MN (US)

(21) Appl. No.: 18/692,146

(22) PCT Filed: Sep. 16, 2022
(86) PCT No.: PCT/US2022/043839

§ 371 Date: Mar. 14, 2024

Related U.S. Application Data
(60) Provisional application No. 63/244,991, filed on Sep. 16, 2021.

(51) Int. Cl. C12N 5/071; A61K 35/32; A61P 19/02

(52) U.S. Cl. CPC C12N 5/0697; A61K 35/32; A61P 19/02; C12N 2500/30; C12N 2506/45

(57) ABSTRACT

A method for producing a multi-tissue organoid generally includes obtaining pluripotent stem cells (PSCs) and inducing growth of the multi-tissue organoid by culturing the harvested PSCs in suspension in a culture vessel. Another method for producing a multi-tissue organoid generally includes introducing a pluripotent stem cell into a cell culture medium comprising hyaluronic acid, transferring the pluripotent stem cell to a cell culture device that does not include a three-dimensional matrix, culturing the pluripotent stem cell in the cell culture device for at least 1 week, and producing a multi-tissue organoid comprising cartilage, bone, fibrous connective tissue, brain tissue, or epithelial tissue, or a combination thereof.

### CROSS-REFERENCE TO RELATED APPLICATION

This application claims the benefit of U.S. Provisional Patent Application No. 63/244,991, filed Sep. 16, 2021, which is incorporated herein by reference in its entirety.

### SUMMARY

This disclosure describes, in one aspect, methods for producing a multi-tissue organoid (MTO). Generally, the method includes obtaining pluripotent stem cells (PSCs) and inducing growth of the multi-tissue organoid by culturing the harvested PSCs in suspension in a culture vessel.

In another aspect, this disclosure describes an alternative method of producing a multi-tissue organoid (MTO). Generally, the method includes introducing a pluripotent stem cell into a cell culture medium that includes hyaluronic acid, transferring the pluripotent stem cell to a cell culture device that does not include a three-dimensional matrix, culturing the pluripotent stem cell in the cell culture device for at least 1 week, and producing a multi-tissue organoid that includes cartilage, bone, fibrous connective tissue, brain tissue, or epithelial tissue, or a combination thereof.

In one or more embodiments of either method, the cell culture medium includes at least one of hyaluronic acid, fibroblast growth factor, transforming growth factor beta (TGFβ), growth differentiation factor 5 (GDF-5), DMEM/F12, L-ascorbic acid-2-phosphate magnesium, sodium selenium, insulin, NaHCO₃, transferrin, TGFβ1, NODAL or bone morphogenetic protein 2 (BMP-2).

In one or more embodiments of either method, the cell culture device includes a second cell culture medium.

In one or more embodiments of either method, culturing the cell in the cell culture device includes culturing the cell at room temperature.

In one or more embodiments of either method, culturing the cell in the cell culture device includes culturing the cell at 37° C.

In one or more embodiments of either method, the PSCs are incubated in a bioreactor.

In one or more embodiments of either method, the organoid includes cartilage, bone, fibrous connective tissue, or a combination thereof.

In one or more embodiments of either method, the organoid includes neuronal cells or neuronal tissue.

In one or more embodiments of either method, the method further includes isolating the organoid.

In one or more embodiments of either method, the method further includes dis-aggregating the cells of the organoid to produce a population of individualized cells. In one or more of these embodiments, the dis-aggregated cells include chondrocytes. In one or more of these embodiments of either method, the method further includes allowing the chondrocytes to form a chondrosphere.

In one or more embodiments of either method, the organoid includes: a cell expressing transforming growth factor β1 (TGFβ1); a cell expressing fibroblast growth factor 2 (FGF2); a cell expressing bone morphogenic protein 2 (BMP2); a cell expressing bone morphogenic protein 6 (BMP6); a cell expressing growth differentiation factor 5 (GDF5); a cell expressing secreted frizzled related protein 1 (SFRP1); a cell expressing inhibin subunit βA (INHβA); a cell expressing transforming growth factor β3 (TGFβ3); a cell expressing insulin-like growth factor 2 (IGF2); a cell expressing leukemia inhibitory factor (LIF); a cell expressing bone morphogenic protein 4 (BMP4); a cell expressing BMP endothelial cell precursor-derived regulator (BMPER); or a cell expressing left-right determination factor 1 (LEFTY1); or a combination thereof.

In one or more embodiments of either method, the pluripotent stem cell is an induced pluripotent stem cell (iPSC).

In one or more embodiments of either method, the culture vessel or cell culture device lacks a biomimetic coating.

In another aspect, this disclosure describes a method of treating a subject having, or at risk of having a condition that includes degeneration of articular cartilage. Generally, the method includes administering to the subject a composition that includes chondrogenic MTO-derived material in an amount effective to ameliorate at least one symptom or clinical sign of the condition.

In one or more embodiments, the MTO-derived material includes a chondrocyte, a chondrosphere, or both.

In one or more embodiments the chondrogenic MITO-derived material is administered in an amount effective to promote regeneration of hyaline cartilage, promote production of Type II collagen, decrease the presence of osteophytes, reduce joint pain, reduce joint inflammation, or any combination of two or more of the foregoing.

In one or more embodiments, the condition is osteoarthritis, cartilage injury, intervertebral disc disease, rheumatoid arthritis, hemochromatosis, psoriatic arthritis, gout, axial spondylarthritis, or juvenile arthritis, Saldino achondrogenesis, hypochondrogenesis, platyspondylic lethal skeletal dysplasia, Torrance type Spondyloepiphyseal dysplasia congenita, Kniest dysplasia, SED with metatarsal shortening, Czech dysplasia, Spondyloperipheral dysplasia, Spondyloepirnetaphyseal dysplasia (SEMD), Strudwick type, Stickler syndrome type 1, Mild SED with premature onset arthrosis, Osteochondritis dissecans, Relapsing polychondritis, Chondrocalcinosis, osteochondroma, enchondroma, periosteal chondroma, multiple chondromatosis, enchondromatosis, chondroblastoma, chondromyxoid fibroma, rheumatoid arthritis, uvenile idiopathic arthritis, gout, systemic lupus erythematosus, seronegative spondyloarthropathies, or temporomandibular arthritis.

In another aspect, this disclosure describes a method of treating a subject having, or at risk of having a condition that includes degeneration of degeneration of neuronal cells or neuronal tissue. Generally, the method includes administering to the subject a composition that includes neuronal MTO-derived material in an amount effective to ameliorate at least one symptom or clinical sign of the condition.

In one or more embodiments, the neuronal MTO-derived material is administered in an amount effective to promote formation of neural rosettes in the subject, promote formation of neural progenitors in the subject, increase dopaminergic neurons in the subject, increase mature astrocytes in the subject, increase oligodendrocytes in the subject, increase markers of cerebral cortex formation in the subject, promote engraftment of the transplanted cells within brain tissue of the subject, increase migration of the transplanted cells within brain tissue of the subject, decrease the severity and/or extent of a symptom or clinical sign of a neurological disorder, or any two or more of the foregoing.

In one or more embodiments, the condition is Parkinson's Disease, Alzheimer's disease, amyotrophic lateral sclerosis, Friedreich ataxia, Huntington's disease, Levy body disease, spinal muscular atrophy, progressive supranuclear palsy, multiple system atrophy, or stroke.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Histological characterization of hyaline cartilage formation in multi-tissue organoids (MTOs). Histology of multi-tissue organoids (MTOs) derived from the 1024 induced pluripotent stem cell (iPSC) line at 8 and 30 weeks, and MTOs derived from the 9-1 iPSC cell line (Lindborg et al., Stem Cells Transl Med 5(7):970-979 (2016)) at 11 weeks. (A) Multi-tissue organoid (MTO) derived from the 1024 iPSC cell line at eight weeks shows a developing cartilage nodule with diffuse Alcian blue staining (blue) of early cartilaginous matrix, diffuse labeling for aggrecan (ACAN, brown) in cells and matrix, and type II collagen labeling (COL2A1, brown) in the cartilaginous matrix of a central region of maturing cartilage. Size bars=200 am. (B) MTO derived from the 9-1 iPSC cell line at II weeks shows maturing cartilage with increased Alcian blue positive cartilaginous matrix separating chondrocytes, moderate diffuse staining for type II collagen and diffuse aggrecan labeling. Size bars=50 μm. (C) MTO derived from the 1024 iPSC cell line after 30 weeks in culture show further maturation to hyaline cartilage morphology with chondrocytes surrounded by abundant matrix with diffuse Alcian blue and type II collagen staining, and pericellular aggrecan staining. Size bars=50 μm. (D) Low magnification views MTO derived from the 1024 iPSC cell line at 30 weeks shown in (C), demonstrating the large size attained by some chondrogenic nodules. Measure bar on H&E panel=4,363 μm (4.363 mm). Size bars=1000 μm, (F) Histomorphometric measurements on histological sections of MTOs derived from the 1024 iPSC cell line using aggrecan area fraction (percentage) in MTOs at week 8 and week 11 (**, p=0.002).

FIG. 2. Cartilage development in MTOs is associated with favored bone morphogenic protein (BMP) signaling pathways and increased mesodermal gene expression. (A) Principal component analysis (PCA) of MTO global transcriptomes at 8 weeks, 11 weeks, and 15 weeks. (B) Differential gene expression analysis showing differentially expressed cartilage marker genes between week 8 and week 15. (C) Ontology enrichment of differentially decreased genes between week 8 and week 15.

FIG. 3. Cartilage development in MTOs is associated with favored BMP signaling pathways and increased mesodermal gene expression. (A) Ontology enrichment of differentially increased genes between week 8 and week 15. (B) Table displaying the comparison and statistical results of the overall expression of grouped genes between week 8 and week 15. The number of genes expressed/genes associated with the GO term (retrieved by biomaRt v2.46.3); one-tailed Wilcoxon test comparing normalized and log-transformed transcript counts between week 8 and week 15. *, p-value<0.05; **, p-value<0.01, ***, p-value<0.001, ****, p-value<0.0001.

FIG. 4. Cartilage development in MTOs is associated with favored BMP signaling pathways and increased mesodermal gene expression. Expression of genes in MTOs at week 8, week 11, and week 15 encoding molecules commonly used for inducing chondrogenesis in vitro.

FIG. 5. Graphic summary (non-grey objects) of findings and relevant pathways.

FIG. 6. Cartilage development in MTOs is associated with distinct Wnt and TGF-beta/BMP signaling. 125 genes were selected (FDR<0.05) and their scaled temporal expression at week 8, week 11, and week 15 were plotted and gene expression between week 8 and week 15 was compared. ****, p<0.0001. (left); genes were arranged by decreased expression (upper left) and increased expression (lower left). Functional association network of protein production expressed by genes with decreased expression (upper right). Functional association network of protein production expressed by genes with increased expression (lower right).

FIG. 7. Transcription signatures in MTOs are comparable with human lower limb chondrocytes. (A) Principal component analysis (PCA) on 325 chondrocyte-specific genes in MTOs and human lower limb chondrocytes. (B) Pearson correlation plot of 325 chondrocyte-specific genes in MTOs and human lower limb chondrocytes.

FIG. 8. Transcription signatures in MTOs are comparable with human lower limb chondrocytes. Representative comparisons of marker transcripts (COL2A1, COL9A, COL6A2, COL11A1, COL10A1, MMP13, ACAN, CD44, PGR4) in MTO and human lower limb chondrocytes. MTO, multi-tissue organoid; GPC, growth plate chondrocyte.

FIG. 9. Rat model of osteoarthritis. Hematoxylin and eosin staining of knee joints treated or untreated with organoid-derived chondrocytes. Top image (untreated control) and bottom image (treated with chondrogenic organoids prepared without hydrogel, were scored according to the Osteoarthritis Research Society International (OARSI) method using a scale of 0-12 with 12 indicating severe osteoarthritis FIG. 10. Chondrocyte engraftment in rat model of Osteoarthritis Antibodies specific for human Ku80 (arrows) confirm engraftment of human organoid-derived chondrocytes in the treated rat osteoarthritis model. Control joints showed no Ku80 staining.

FIG. 11. Goat Model of Cartilage Injury Repair. A cartilage injury was surgically induced by full thickness injuries, 8 mm diameter, in a non-weight-bearing area of articular cartilage of stifle (knee) joints in two goats. At surgery, lesions were treated as follows. In goat #1 (male), the treated side received single-cell chondrocyte product derived from MTO-derived chondrospheres embedded in fibrin glue (TISSEEL, Baxter International Inc., Deerfield, IL), and the control side received only fibrin glue. In goat #2 (female), the treated side received MTO-derived chondrospheres embedded in fibrin glue and the control side only received fibrin glue. 12 weeks after surgery the goats were sacrificed, and knees were examined by gross pathology before they were fixed in Neutral Buffered Formalin (NBF) and submitted for histopathologic examination. Gross pathology (left) and Hematoxylin and Eosin staining (right) show increased growth in treated lesions compared to control joints. These results indicate prominent neocartilage formation in treated lesions and minimal or no regeneration in control lesions.

FIG. 12. A cartilage injury was surgically induced by full thickness injuries, 8 mm diameter, in a non-weight-bearing area of articular cartilage of stifle (knee) joints in two goats. At surgery, lesions were treated as follows. In goat #1 (male), the treated side received single-cell chondrocyte product derived from MTO-derived chondrospheres which were embedded in fibrin glue (TISSEEL, Baxter International Inc., Deerfield, IL), and the control side received only fibrin glue. In goat #2 (female), the treated side received MTO-derived chondrospheres which were embedded in fibrin glue and the control side only received fibrin glue. 12 weeks after surgery the goats were sacrificed, and knees were examined by gross pathology before they were fixed in Neutral Buffered Formalin (NBF) and submitted for histopathologic examination. Alcian blue staining tissue (upper panel-blue) demonstrates the proteoglycan/hyaluronic acid components typical of cartilage. Type II Collagen Immunohistochemistry (lower panel-brown) demonstrates cartilage also showed prominent amounts of the specific cartilage marker protein, type II collagen, in the regenerating articular cartilage in the treated joints of both subjects. These results indicate prominent neocartilage formation in treated lesions and minimal or no regeneration in control lesions.

FIG. 13. Histology of week 2 MTO showing neural rosettes (arrows) in top left panel, neural tube-like structure (arrows) in upper middle panel, and organizing neuroepithelial region in upper right panel (star). Lower panel shows extensive labeling of an MTO with the neural marker β-3 tubulin.

FIG. 14. Immunofluorescent staining of week 2 MTO. Neural rosettes (arrows) show cells with double immunofluorescent labeling for SOX1/Nestin and SOX2/Nestin consistent with neural progenitor/stem cells present in the rosettes.

FIG. 15. Immunohistochemistry of 2 week MTO. Immunohistochemistry stains show expression of several neural markers (brown staining areas) including beta-3 tubulin (0-3 tubulin), Sox2, Nestin, Nurr1, and Pax6. Tyrosine hydroxylase is also shown (arrows), which is a marker for dopaminergic neurons. A hallmark lesion of Parkinson's Disease is loss of dopaminergic neurons in the substantia nigra.

FIG. 16. Immunohistochemistry of 2 week MTO. Immunohistochemistry staining for Olig2. Olig2 is a marker of oligodendrocyte and motor neuron progenitors (brown staining) and is present in the neuroepithelial areas of MTOs at two weeks.

FIG. 17. Immunofluorescent staining of week 4 MTO. Immunofluorescent staining (green) demonstrating three markers of cerebral cortex formation including Tbr1, doublecortin, and reelin. Cell nuclei stained blue with To-Pro-3.

FIG. 18. Immunohistochemistry of histological sections of MTOs beyond week 6. Histologic sections of MTOs stained by immunohistochemistry show further maturation of neural and glial cells which is evidenced by expression of GFAP (red) indicating astrocytes, MAP2 (brown) indicating neurons, and MBP (brown) indicating oligodendrocytes.

FIG. 19. Engraftment sites of six-week-old human MTO-derived cells are demonstrated in two rats at eight weeks post-op with STEM121 immunohistochemistry stains. Brown staining areas indicate positive staining for STEM121 and demonstrate presence of human cells. In high magnification images (right panels), narrow STEM121 positive cell projections can be seen extending into adjacent areas of the rat brains consistent with neural engraftment.

FIG. 20. Engraftment sites of six-week-old human MTO-derived cells are demonstrated in two rats at eight weeks post-op with STEM121 immunohistochemistry stains (brown staining areas indicate positive staining for STEM121) is also present on the contralateral side of the brain from where the cells were injected indicating extensive migration of the transplanted cells within the brain.

FIG. 21. Rat Brain (striatum) eight weeks post-op following transplantation with six-week-old human MTO-derived cell product. (A) Co-localization of myelin basic protein (MBP) (green) and STEM121 (red) indicating engrafted human oligodendrocytes (B) Microtubule-Associated Protein 2 (MAP2) (green) and STEM121 (red) co-localization indicating engraftment of human neurons.

FIG. 22. Therapeutic benefit of six-week-old human MTO-derived neural cells as measured by amphetamine-induced rotations (turns per minute). Nude rats with induced Parkinsonism were treated with 300,000 cells from six-week-old MTO-derived cells by injection into the striatum. Baseline assessment was done prior to MTO cell therapy and assessed on post operative weeks 2, 4, 6, and 8 High rotation turns/min (>10) indicate Parkinsonism. B2 is a normal control (non-Parkinsonian) rat. A therapeutic benefit (reduced rotations) can be seen in rats B4, B7, B8, andB11 over the course of the study (Rat 11 died after week 2 from infection unrelated to the cell product.).

FIG. 23. MTO derived from iPSC line 1024 generated without the use of a 3D matrix, harvested at week 16, and immunohistochemically stained for GFAP as a marker of astrocytes. Red-brown staining cells indicate positive staining.

### DETAILED DESCRIPTION OF ILLUSTRATIVE EMBODIMENTS

This disclosure describes a novel method of growing iPSC-derived multi-tissue organoids (MTO) that results in the MTOs spontaneously producing therapeutic materials. The therapeutic materials include, but are not limited to, chondrocytes that produce hyaline cartilage, chondrospheres, and neural tissues.

Organoids are three-dimensional (3D) cultures that realize the self-organizing potential of stem cells. Transcriptome analyses show that organoids can recapitulate a variety of early developmental processes in human organs, such as brain, retina, kidney, intestinal epithelium, and trophoblast. Since the highest levels of chondrogenic tissue formation occur during fetal development and early stages of life, 3D organoid systems are uniquely able to in vitro chondrogenesis and associated organization of extracellular matrices. Human clinical translation of organoid-derived hyaline cartilage and chondrocytes requires xenobiotic-free and serum-free culturing protocols. Elucidation of key mesoderm formation pathways associated with cartilage production in organoids is needed to facilitate future cartilage production through organoid engineering.

### Exemplary Therapeutic Areas of Use

### Osteoarthritis

One area of potential clinical utility for MTO-derived materials is treating osteoarthritis. The exact pathogenic mechanisms of osteoarthritis remain unclear. Osteoarthritis was initially thought of as an unavoidable passive age-related disease caused by biomechanical factors. It is now widely accepted as a dynamic and multifaceted process, involving inflammatory, mechanical, and metabolic factors that create an imbalance between the destruction and repair of joint tissues, resulting in the inability of the articular surface to absorb and distribute the mechanical load through the synovial joint. Osteoarthritis is clinically manifested by synovial distension and inflammation, thin and rough articular cartilage, and/or reactive bone hyperplasia at the joint edge and beneath the cartilage. Radiographically, joint space narrowing (JSN), osteophytosis, subchondral sclerosis, cyst formation, and/or abnormalities of bone contour also may be seen. Osteoarthritis is not restricted to the cartilage or subchondral bone; rather, it results from interplay among tissues of the osteochondral complex, including adipose and synovial tissue, as well as the ligaments, tendon, and muscles that surround the joint. However, there are no disease modifying osteoarthritis drugs that have been shown to control or reverse the disease.

iPSC-derived MTOs provide the potential for a disease modifying therapy for osteoarthritis that will repair and restore cartilage, reduce inflammation, and/or significantly delay or reduce the need for joint replacement.

### Cartilage Injury Repair

Another area of clinical utility for MTO-derived materials is repair of cartilage injury. Cartilage tears and osteochondral defects involving the articular cartilage are common injuries that may lead to joint replacement. Injured articular cartilage has a limited capacity for self-regeneration and treatment options for symptomatic cartilage lesions are limited. Surgical repair methods (e.g., osteochondral auto/allograft transplantation and bone marrow stimulation via microfracture) all have limitations including the scarcity of suitable grafts and/or their inability to yield repair tissues composed of hyaline cartilage rather than fibrocartilage. Cell-based methods for treating articular cartilage injuries have been explored to avoid the limitations of surgical repair methods. First-generation and second-generation autologous chondrocyte implantation methods have yielded good patient outcomes, but the morbidity and cost associated with the surgical harvest of autologous chondrocytes for in vitro expansion remain limitations for the widespread clinical use of chondrocyte implantation methods.

In vitro chondrogenesis and hyaline cartilage production from human stem cells have been explored to avoid the limitations associated with the surgical harvest of autologous chondrocytes.

### Neurodegenerative Disorders

Yet another area of clinical utility for MTO-derived materials is treating chronic and/or progressive neurological disorders such as, for example, Parkinson's Disease. Affected people typically have hand tremors, rigidity, slowness of movement and postural instability. As the disease progresses the clinical signs worsen to include festination (rapid shuffling steps and a forward-flexed posture when walking), difficulty in speaking (slurred speech), swallowing difficulty, a mask-like facial expression, and cognitive decline. Patients are eventually bed-ridden, being unable to move or care for themselves, and incur high inpatient care and nursing home costs. Currently available treatments for Parkinson's Disease (drugs and deep brain stimulation) attempt to compensate for the loss of dopamine (DA) by either providing the chemical precursor or by stimulating those brain regions that directly respond to the dopamine-producing midbrain cells. Unfortunately, these therapies are mainly palliative, eventually lose efficacy, can be associated with significant side effects, and do not prevent disease progression.

### Preparing and Characterizing Multi-Tissue Organoids

Induced pluripotent stem cells (iPSCs) have the potential to differentiate into chondrocytes; however, clinical translation of iPSC-derived chondrocytes still faces several challenges. The common approach for generating iPSC-derived chondrocytes has been to use a 2D-3D sequential culture where iPSC-derived mesodermal cells are cultured in monolayer before being transferred to three-dimensional cell culture. Implementing three-dimensional culture into the process improves the quality of the cartilage generated. Nevertheless, these existing step-wise protocols are labor-intensive, involve the use of fetal bovine serum (FBS), and involve manipulations of inductive and repressive signals for mesoderm specification in embryonic development. Moreover, the long-term maintenance and consequences of chondrocytes in suspension and pellet cell cultures remain to be explored.

### Organoid-Derived Chondrogenic Materials

To gain a better understanding of the molecular pathways in MTOs during in vitro cartilage production, RNA-seq was performed at eight weeks, 11 weeks, and 15 weeks following MTO induction. Comparing the RNA-seq data against existing RNA-seq data obtained from human chondrocytes at different life stages showed that relevant gene expression in week-15 MTOs strongly correlated with human fetal lower limb tissues.

### Chemically Defined and Xeno-Free Culture

In one aspect, this disclosure describes the spontaneous emergence of human hyaline cartilage from hiPSC-derived MTO cultured with a xeno-free and feeder-free protocol. As used herein, the term “xeno-free” refers to a culture system designed to include components derived only from the same species as the iPSCs. Thus, while described herein in the context of an exemplary embodiments in which human hyaline cartilage is produced by an MTO derived from human iPSCs (hiPSCs) in a xeno-free system that includes only human components, xeno-free systems based on using iPSCs from other species will be limited to having components only from the species from which the iPSCs are obtained. As used herein, the term “feeder-free” refers to a culture system without the use of feeder cells, which are typically fibroblasts.

### Overcoming Limitations to Previous Method

As mentioned above, methods for generating cerebral organoids (CO) from human pluripotent stem cells using a chemically defined hydrogel material (CELL-MATE3D, BRTI Life Sciences, Two Harbors, MN) and culture medium (E8), and characterization of the organoids have been previously reported (Lindborg et al., Stem Cells Transl Med 5(7):970-979 (2016); U.S. Patent Application Publication No. US 2020/0248139 A1). The culture time span of these organoids was limited due to central necrosis of the organoids when they reached 2-3 mm in diameter, presumably due to hypoxia.

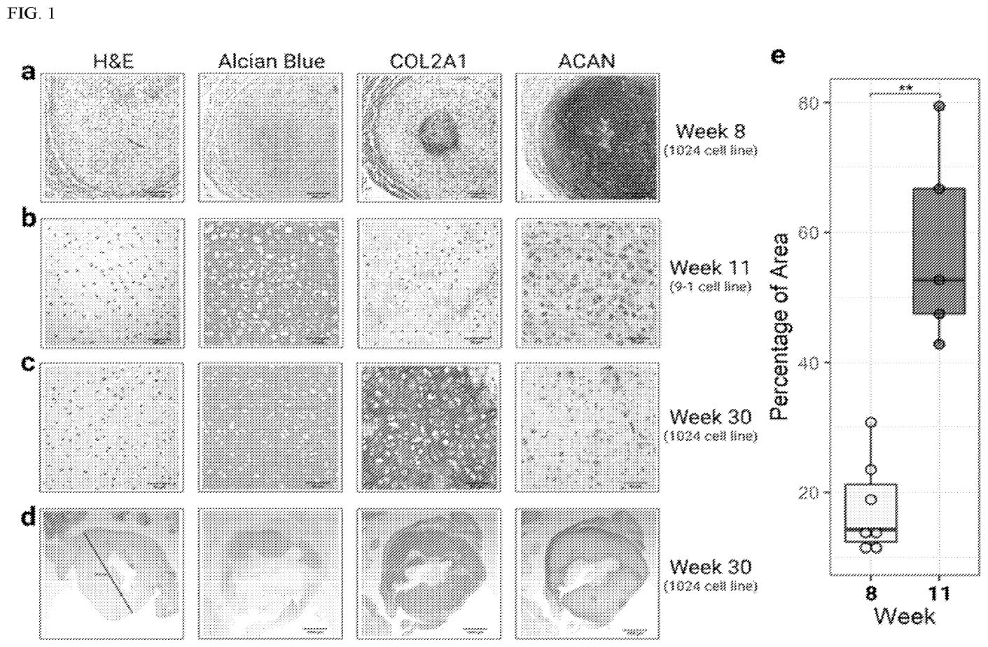

To address this limitation, this disclosure describes methods using a bioreactor system that has a gas-permeable bottom (G-REX 100, Wilson Wolf, New Brighton, MN), thereby allowing oxygen diffusion from the bottom as well as the top of the culture medium interfaces. Using this system along with continued use of only E8 medium, one can routinely culture organoids for months (e.g., up to 30 weeks). The method described herein further modifies the previously reported methods by eliminating the use of the chitosan component of CELL-MATE3D (BRTI Life Sciences, Two Harbors, MN). Cartilage-like tissues emerged as even though the cerebral phenotype of the organoids was still prominent. Cartilage, which formed centrally in the MTOs, was easily recognizable through histology due to its distinct morphology and characteristic histochemical and immunohistochemical features. The main types of cartilage—articular, hypertrophic, elastic, and fibrous cartilage—can be distinguished by the structure and composition of their extracellular matrix (ECM). Articular cartilage, for example, has a hyaline rather than fibrous morphology (fibrocartilage), and contains predominantly type II collagen, little or no type I collagen (fibrocartilage) or type X collagen (hypertrophic cartilage), and no elastic fibers. By week 8, hyaline cartilage became very distinguishable in MTOs, as indicated by the development of characteristic, abundant, homogenous pale basophilic extracellular matrix (ECM) in H&E-stained sections (FIG. 1A) and prominent Alcian blue staining of proteoglycan/hyaluronic acid components typical of cartilage ECM (FIG. 1B). Immunohistochemistry of MTO cartilage showed increasing amounts of type II collagen over time as the cartilage matured and showed extensive expression of aggrecan at all time points (FIG. 1C, D). Type VI collagen was also extensively expressed in MTO cartilage while Type I collagen showed expression in some sites at the periphery of MTO cartilage, and Type X collagen generally showed no immunoreactivity above background. The hyaline cartilage phenotype was stably maintained from week 8 to week 30 with prominent growth in size (FIG. 1A-D). To further assess this, histomorphometric measurements were performed on MTO histologic sections using aggrecan area fraction as a global indicator of developing and mature cartilage composition of the MTOs. At week 8 (n=7 biological replicates) aggrecan area fraction was 17.7% (±7.2) and at week 11 (n=5) was 57.8 (±15.1) with unpaired t-test showing a significant difference (p=0.0001) (FIG. 1E).

### Global Transcriptome Reveals Signatures of Mesoderm Formation in MTO

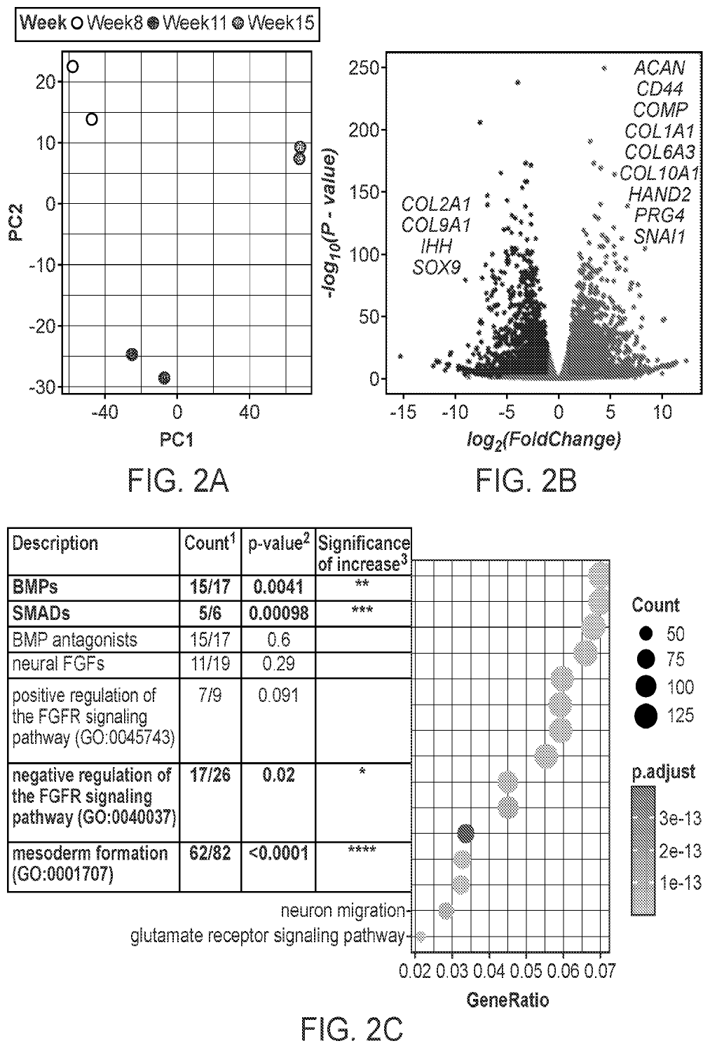

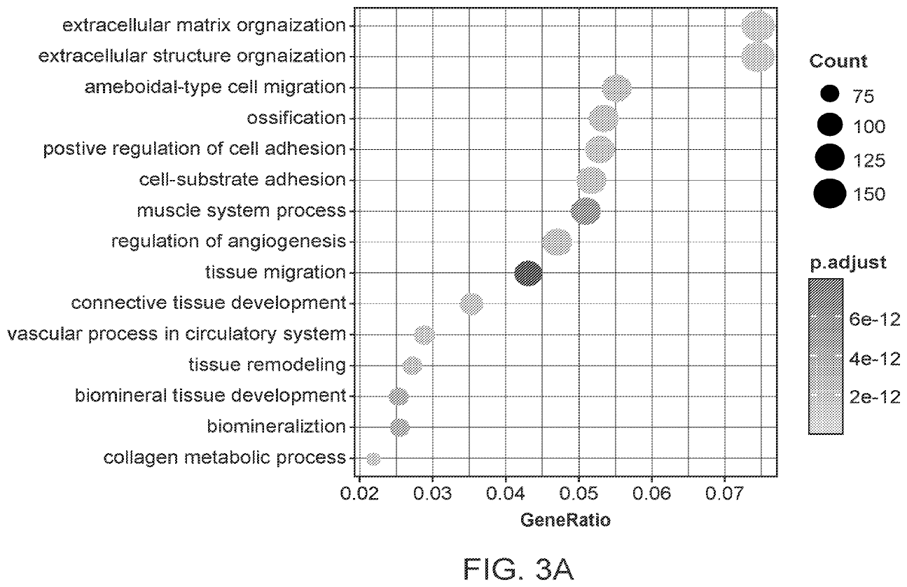

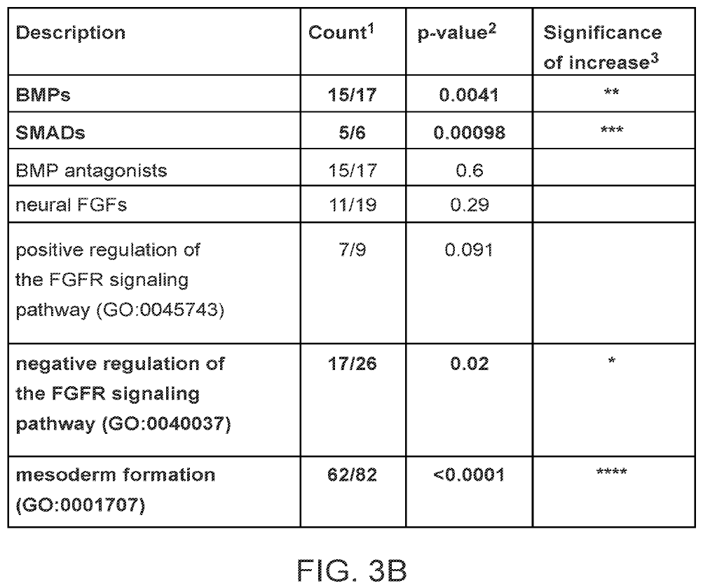

To understand the transcriptome change underlying the phenotypic development of cartilage in MTOs, bulk RNA-seq was performed on 1024-derived MTOs at week 8, week 11, and week 15, which covered the time span during which histologic analysis showed emergence, expansion, and maturation of the MTO cartilage. Principal component analysis (PCA) of MTO global RNA expression data showed distinct clustering corresponding to the time of collection (FIG. 2A). Differential gene expression and gene ontology (GO) enrichment of differentially expressed genes was performed for all three comparisons. Although COL2A1 content increased in MTO cartilage (FIG. 1C), it showed decreased expression in bulk RNA-seq, perhaps due to the multi-tissue nature of MTO. Other cartilage markers, however, such as ACAN, CD44, (COMP, PRG4, and SNAl1, displayed significantly increased expression over the time course of the experiments (FIG. 2B). Although transcript levels for collagen type I/X increased, the expression of another hypertrophic marker, IHH (FIG. 2B), decreased. Additionally, type I collagen is the most abundant collagen and its expression is not limited to cartilage. Therefore, increased levels of type I collagen transcripts in MTO does not necessarily reflect an increase of type I collagen in the composition of MTO cartilage. Interestingly, consistent downregulation of neural processes, such as synapse organization and axonogenesis, and upregulation of mesodermal processes, for example, extracellular matrix organization, connective tissue development, and cartilage development were observed (FIG. 2C; FIG. 3A).

Interplays between bone morphogenetic protein (BMP) and fibroblast growth factor (FGF) signaling play a highly conserved role in processes including, but not limited to, neural induction and mesoderm patterning, chondrocyte differentiation and proliferation, and endochondral ossification during embryonic development. The gradual increase in cartilage production observed from week 8 to week 15 suggests that the global neural-to-mesodermal transition observed in human MTO may be associated with significantly altered dynamics between BMP and FGF pathways. Specifically, BMP pathways may be favored, which would entail increased expression of components in the BMP signaling pathways, reduced or unchanged levels of BMP antagonists, and/or reduced or steady levels of FGF signaling Pathways during mesoderm development processes are intertwined and expressed in a variety of tissues, identifying and examining only a few expressed genes in one pathway may be biased in bulk-sequencing of MTOs. Therefore, the expression of genes was examined comprehensively based on previous publications and GO terms and the overall expression of grouped genes were compared between week 8 and week 15. A significant increase in the overall expression of BMPs (p=0.0041) and their intracellular signaling transducers—SMADs (p=0.00098) (FIG. 3B)—occurred. The overall expression of BMP antagonists remained unchanged (p=0.6) (FIG. 3B). As for FGF signaling pathways, the expression of neural FGFs was examined, which were expressed at lower levels in comparison to other transcripts described previously although they did not change significantly (p=0.29) (FIG. 3B). Further, the dynamic expression of other components in the FGFR pathways was examined, revealing a significant increase (p=0.02) in negative regulation of the FGFR signaling pathway (GO:0040037) and a decrease—although not statistically significant (p=0.097)—in the expression of genes involved in the positive regulation of the FGFR signaling pathway (GO:0045743) (FIG. 3B). This suggests that neural FGFs and their downstream pathways were suppressed, agreeing with the observed histological decrease in neural components. Finally, the expression dynamics of genes involved in mesoderm formation (GO:0001707) were examined, revealing a significantly increased (p<0.0001) expression of genes involved in this biological process (FIG. 3B).

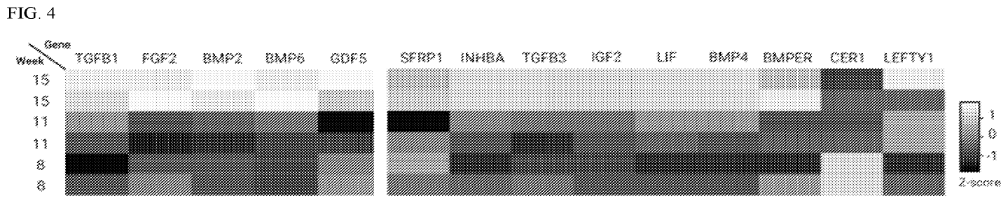

Because the MTOs spontaneously favored the phenotype of cartilage production without the addition of chemicals beyond E8 medium, MTOs may intrinsically increase the expression of differentiation factors that are used to induce chondrogenesis. Expression of BMP2, BMP6, FGF2, and TGFB1 significantly increased in MTOs spontaneously from week 8 to week 15 (FIG. 4) In addition, the expression of other genes whose products were used for chondrogenic differentiation in other protocols we investigated, including TGFB3, INHBA, BMPER, BMP4, LIF, IGF2, LEFY1, SFRP1, and CER1. All but CER1 displayed increased patterns of expression (FIG. 4). This indicates that molecular mechanisms underlying spontaneous chondrogenesis in MTOs may resemble those observed in hiPSC-induced chondrogenic differentiation.

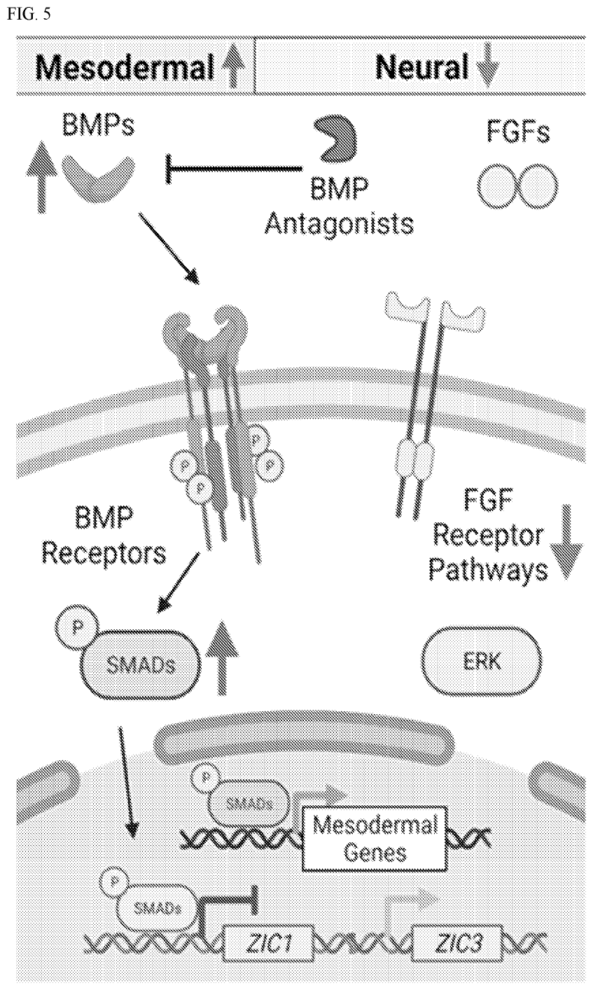

Overall, the global transcriptome of hiPSC-derived MTOs showed that increased gene expression in BMP signaling and mesoderm development as well as decreased gene expressions in neural FGF signaling were associated with the increased cartilage production in MTOs from week 8 to week 15 (FIG. 5).

Distinct Signaling Pathways Associated with Increased Articular Cartilage Development in MTOs

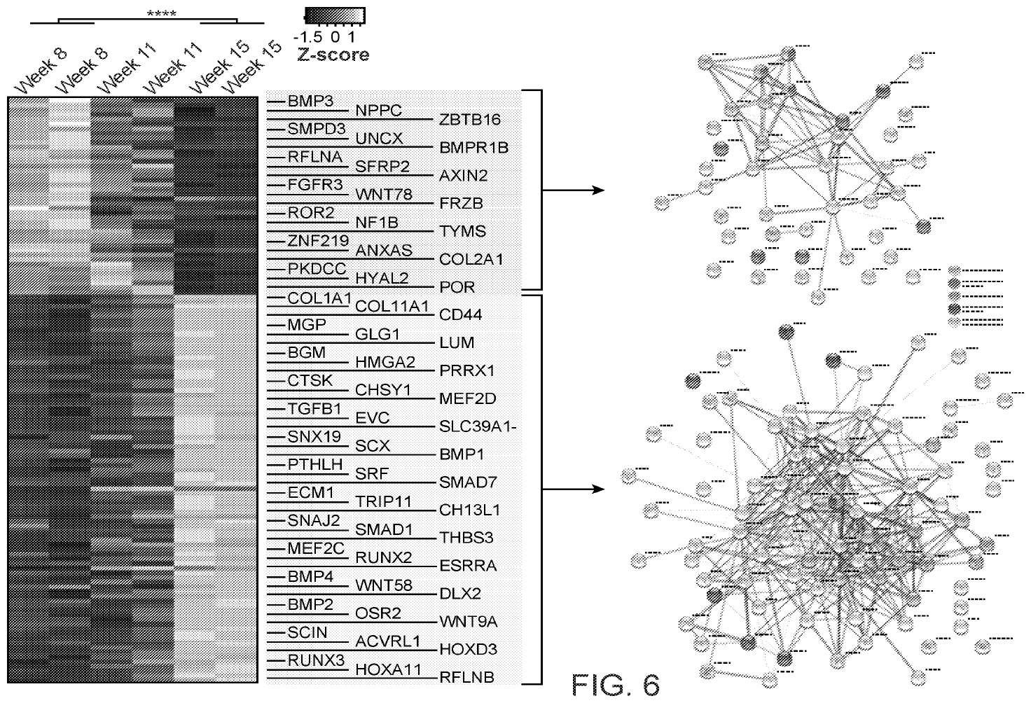

The establishment of both cartilage and bone formation is the result of chondrogenesis, which plays an essential role during the fetal development of the mammalian skeletal system. Hypertrophy of chondrocytes and deterioration of cartilage matrix precede endochondral ossification that leads to the formation and growth of long bones. Gene expression for chondrogenesis and ossification overlap greatly due to the unified nature of cartilage and bone formation. As previously mentioned, an increase in several cartilage marker genes as well as contradicting expression of IHH and COL10A1 in the MTOs was observed (FIG. 1). No deterioration, mineralization, or osteogenesis of the cartilage matrix was observed, however, even up to 30 weeks. Therefore, the temporal expression patterns of genes involved in cartilage development were investigated to determine whether signaling pathways that may contribute to the long-term maintenance of chondrocytes could be identified. To achieve this, gene expression under the GO term cartilage development (00:0051216), which includes both the positive and negative regulators of cartilage development, was investigated first. The expression pattern of 125 (FDR<0.05) out of the 194 genes retrieved by biomaRt were visualized, showing a clear separation of 42 with decreased expression and 83 genes with increased expression (FIG. 6). In addition, despite the decrease of 42 genes, the overall gene expression levels increased significantly (p<0.0001) for cartilage development (FIG. 3, left panel), agreeing with the expansion of cartilage in MTOs.

Next, STRING was used to construct two functional association networks consisting of proteins expressed by the described genes to identify distinct interactions of gene products between selected genes with increased and decreased expression. Transcription factors and growth factors were the most connected molecules for both networks. Wnt signaling molecules constituted the most prominent local network clusters in the network cluster organization for decreased gene products (FIG. 6, upper right panel; (Table 1)).

TABLE 1
Local network cluster (STRING)

| | | | | | |
|---|---|---|---|---|---|
| | | | | | false |
| | | observed | background | | discovery |
| term ID | iterm description | gene count | gene count | strength | rate |

Decrease

| | | | | | |
|---|---|---|---|---|---|
| CL:5705 | frizzled binding, and Wnt- | 5 | 41 | 1.75 | 7.61 × 10⁻⁶ |
| | protein binding | | | | |
| CL:5698 | Wnt signaling pathway, and | 7 | 186 | 1.24 | 9.06 × 10⁻⁶ |
| | TGF-beta signaling pathway | | | | |
| CL:5709 | Wnt, and Wnt-protein | 4 | 30 | 1.79 | 2.25 × 10⁻⁵ |
| | binding | | | | |
| CL:5710 | Negative regulation of TCF- | 3 | 18 | 1.89 | 0.0002 |
| | dependent signaling by | | | | |
| | WNT ligand antagonists, | | | | |
| | and Frizzled/Smoothened, | | | | |
| | transmembrane domain | | | | |

Increase

| | | | | | |
|---|---|---|---|---|---|
| CL:5923 | TGF-beta signaling | 12 | 74 | 1.58 | 4.61 × 10⁻¹³ |
| | pathway, and DAN domain | | | | |
| CL:5698 | Wnt signaling pathway, and | 15 | 186 | 1.28 | 5.03 × 10⁻¹³ |
| | TGF-beta signaling pathway | | | | |
| CL:5925 | TGF-beta signaling | 11 | 64 | 1.61 | 1.04 × 10⁻¹² |
| | pathway, and Regulation of | | | | |
| | signaling by NODAL | | | | |
| CL:5929 | Signaling by BMP, and | 6 | 19 | 1.87 | 3.08 × 10⁻⁸ |
| | BMP receptor binding | | | | |

Wnt signaling cascades have essential roles in the development and homeostasis of chondrogenesis and ossification. In general, canonical Wnt cascades—including ROR2 and SFRP2 highlighted in the network-inhibit the early stages of chondrogenesis. WNT7A overexpression blocked early chondrogenesis in chick limb model. During endochondral ossification, Wnt signaling pathways promote chondrocyte hypertrophy. SFRP2, WNT01B, and WNT7B are known positive regulators of ossification. Additionally, some non-Wnt positive regulators of ossification were also downregulated. Among the upregulated members, clusterings centered on TGF-beta/BMP signaling pathways (FIG. 6, lower right panel). Also, a unique signature of increased downregulation of the canonical Wnt signaling pathway was observed, agreeing with the association network formed by protein products of genes with decreased expression. In contrast to the previous network, several negative regulators of ossification showed little overlap with TGF-beta/BMP signaling pathways (FIG. 6, lower right panel). In summary, from week 8 to week 15, the cartilage in MTOs was likely stably growing due to continued increase in TGF-beta/BMP pathways promoting chondrogenesis and downregulation of Wnt signaling cascades that inhibit chondrocyte hypertrophy and ossification in maturing chondrocytes.

In addition, the expression of genes for biological processes of interest were also compared as described above (Table 2) using GO term gene lists retrieved by biomaRT from GO to reduce bias in gene selection.

TABLE 2
Biological processes (GO)

| | | Count in | | Significance |
|---|---|---|---|---|
| GO Terms | Description | network¹ | p-value² | of increase³ |
| GO:0061036 | positive regulation of cartilage | 29/32 | 0.0053 | ** |
| | development | | | |
| GO:0060591 | chondroblast differentiation | 4/5 | 0.0039 | ** |
| GO:0060536 | cartilage morphogenesis | 8/11 | 0.019 | * |
| GO:0003417 | growth plate cartilage development | 18/18 | 0.058 | |
| GO:0061037 | negative regulation of cartilage | 26/32 | 0.17 | |
| | development | | | |
| GO:0032331 | negative regulation of chondrocyte | 21/25 | 0.17 | |
| | differentiation | | | |
| GO:0003413 | chondrocyte differentiation involved | 15/15 | 0.24 | |
| | in endochondral bone morphogenesis | | | |
| GO:0061975 | articular cartilage development | 3/3 | 0.031 | * |
| GO:0060532 | bronchus cartilage development | 2/2 | 0.8 | |
| GO:0060534 | trachea cartilage development | 6/9 | 0.6 | |

¹number of genes expressed/genes associated with the GO term (retrieved by biomaRt v2.46.3)
²one-tailed Wilcoxon test comparing normalized and log-transformed transcript counts between week 8 and week 15
³*, p-value < 0.05; **, p-value < 0.01

Again, genes in GO terms associated with promoting cartilage maturation, including positive regulation of cartilage development (GO:0061036), chondroblast differentiation (GO:0060591), and cartilage morphogenesis (GO:0060536), demonstrated significantly increased expression (Table 2), while growth plate cartilage development gene set (GO:0003417) was not significantly increased (p=0.058). Moreover, the expression of genes associated with negative regulation of chondrocyte maturation and promotion of endochondral ossification did not show significant changes (Table 2). Since the expression of the growth plate cartilage development (G00003417) marker gene was only moderately increased (p=0.058), the growth in hyaline cartilage produced by MTO may be more closely associated with the increased expression of articular cartilage gene markers but not others. Indeed, a significant increase in the expression of articular cartilage marker genes (GO:0061975; p=0.031), but not bronchus or trachea cartilage development (GO:0060532, GO:0060534), was observed.

To summarize, protein products of genes with decreased expression in MTOs from week 8 to week 15 were clustered around components of Wnt signaling pathways that promote ossification while those with increased expression formed prominent networks around TGF-beta/BMP signaling pathways. Moreover, the observed molecular signaling clusters were associated with a unique increase in the gene expression of articular cartilage development.

### Transcriptomic Comparisons Between MTO Chondrocytes and Lower Limb Chondrocytes Cross Human Life Stages

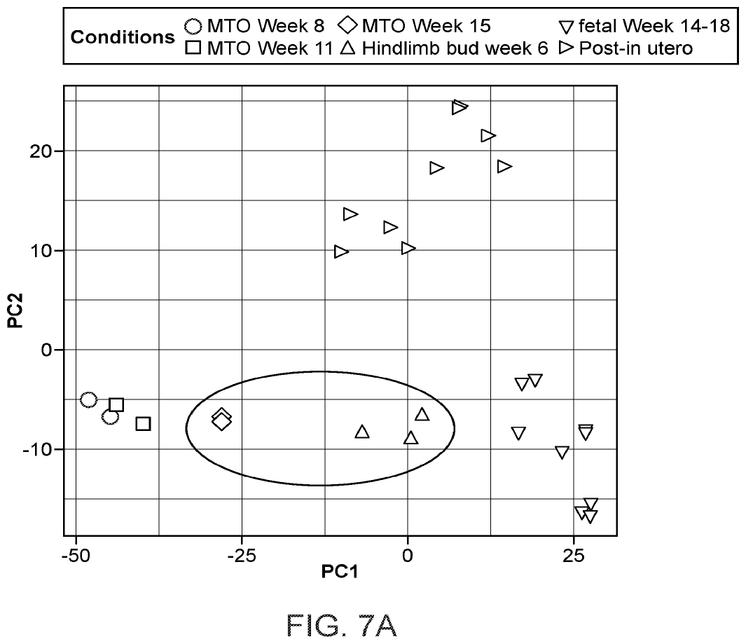

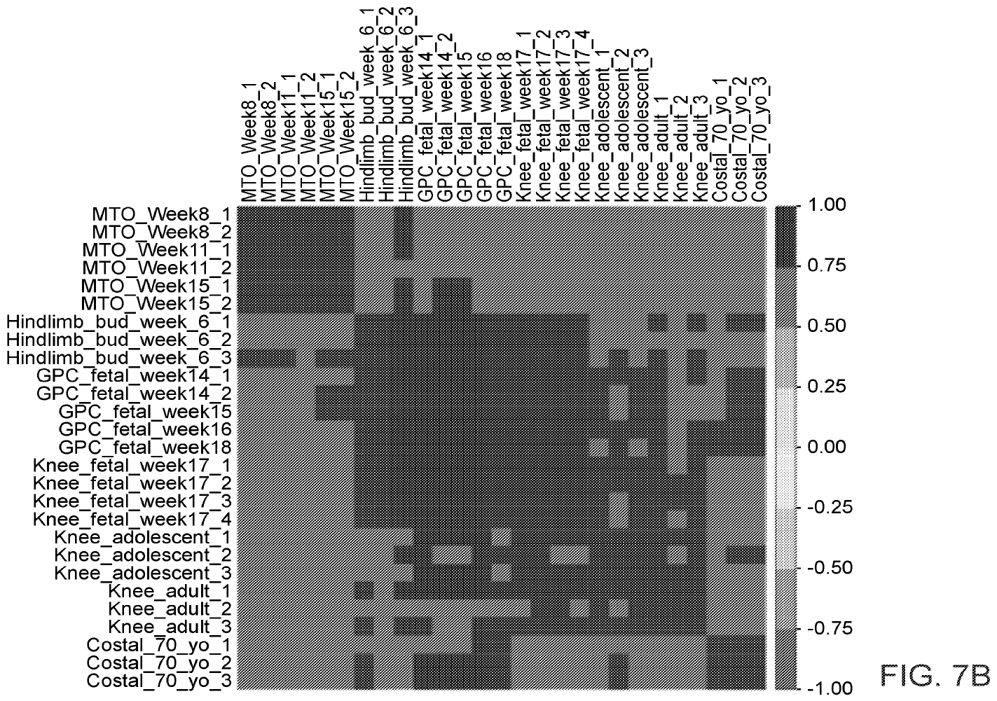

Because MTO RNA-seq revealed a unique increase in articular cartilage development, there may be a strong correlation between MTO chondrocytes and human growth plate chondrocytes and/or articular chondrocytes in the expression of genes specific to human growth plate chondrocytes. Existing RNA-Seq data collected from human embryonic limb bud (week 6, Li et al., BMC Genomics 18(1):983 (2017)), growth plate chondrocytes (week 14, 15, 16, and 18; Li et al., BMC Genomics 18(1):983 (2017)), knee chondrocytes (week 17, adolescent (Hicks et al., Nat Cell Biol 20:46-57 (2018)), and adult (Ferguson et al., Nat Commun 9(1):36³4 (2018))), and costal chondrocytes (˜70-year-old adults) were reprocessed. 325 genes known to be specifically expressed by chondrocytes were validated and principal component analysis (PCA) was performed on these genes. The effect of different studies is minimal as gene expression was clustered by life stages rather than particular studies (FIG. 7A). Specifically, fetal tissues (growth plate and knee chondrocytes) clustered together while cartilage tissues from adolescents, adults, and 70-year-olds (knee and costal chondrocytes)—collectively addressed as post-in utero tissues—located in close proximity to each other (FIG. 7A). 15 week-MTO was most similar to six-week human limb bud cartilaginous tissues (FIG. 7A). Then, the Pearson correlation coefficient was used to examine the correlation among all samples and genes included in the PCA analysis. While all MTOs showed >60% correlation with human chondrocytes and cartilage tissues from all life stages, week 15 MTOs showed an even stronger correlation (>76%) with six-week human limb bud, 14-week fetal growth plate chondrocytes, and 15-week fetal growth plate chondrocytes (FIG. 78). Moreover, MTOs, on average for previously described genes of interest, showed a significantly higher (p<0.0001; 95% Cl [0.053, +∞]) correlation with fetal chondrocytes than post-in utero knee chondrocytes; the average correlation between MTO and fetal tissues was 71% while that for post-in utero tissues was 65%.

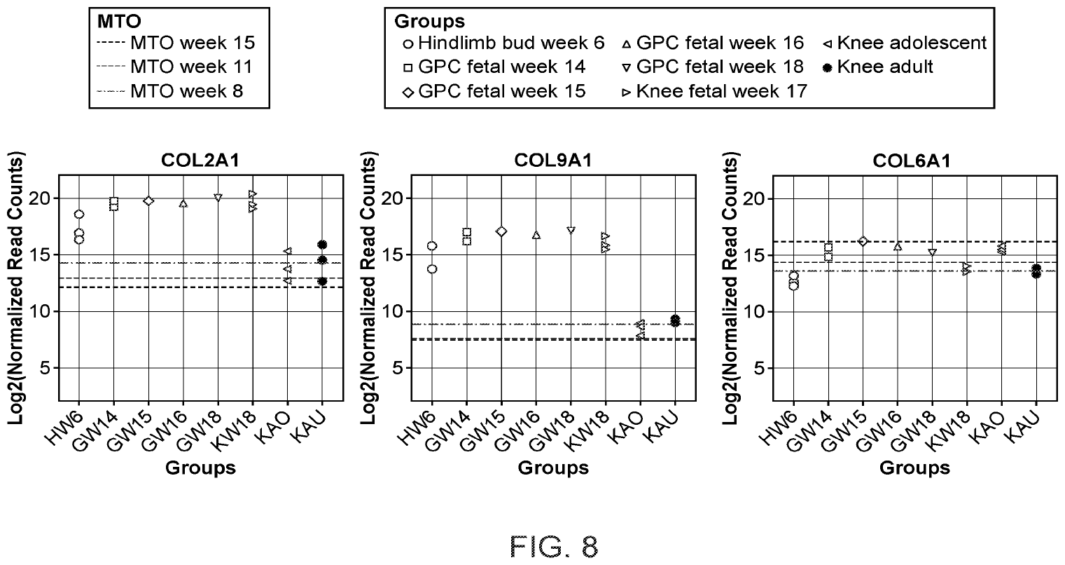

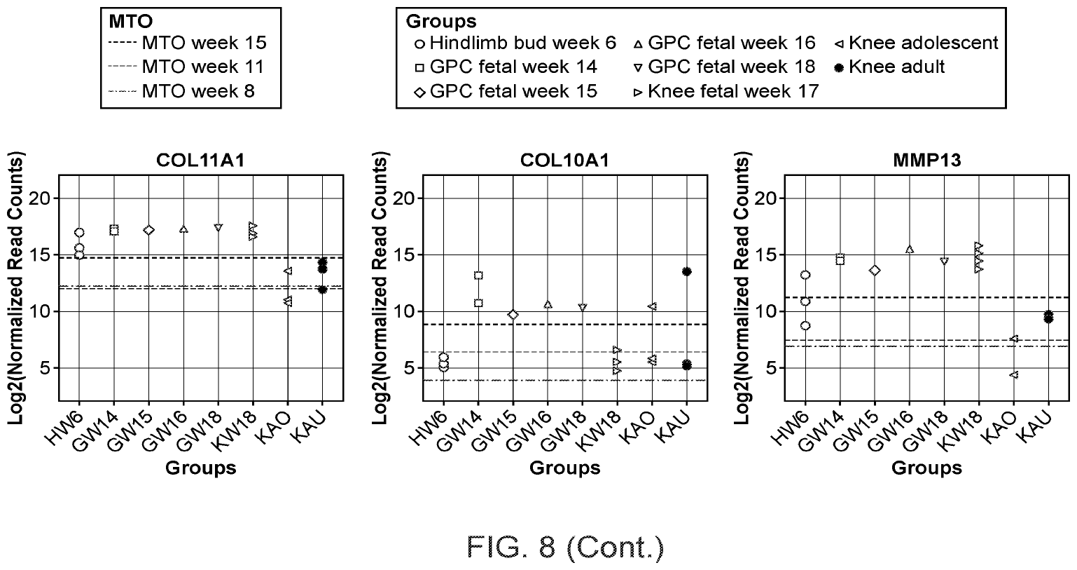

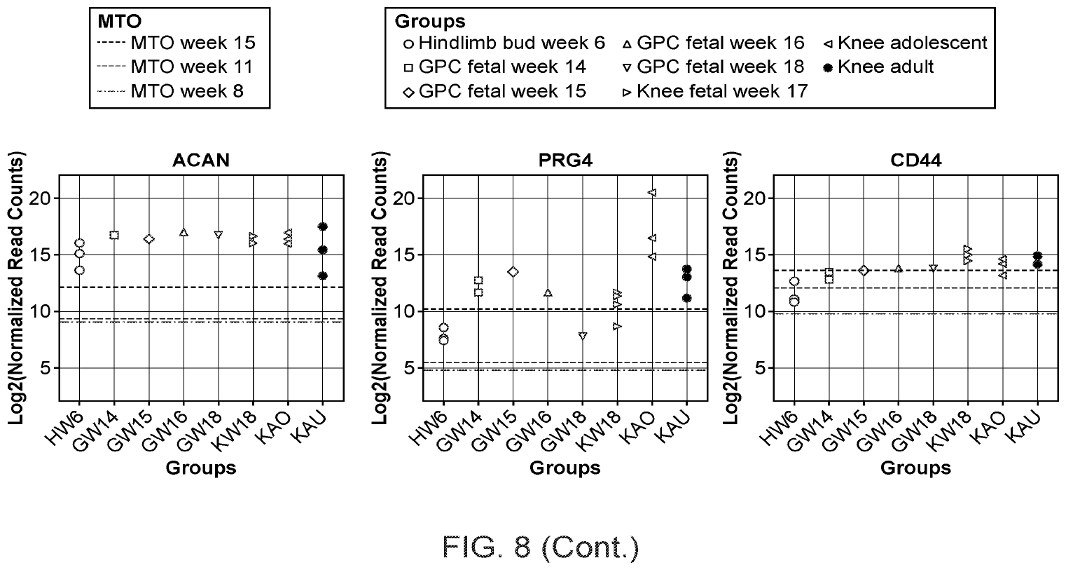

The expression of known collagen genes (COL2A1, COL9A1, COL6A2, COL1A1, COL10A1), hypertrophic markers (COL10A1, MMP13), and some components reflecting the secretory functions of chondrocytes (ACAN, CD44, PGR4) were further examined in MTOs as compared to human lower limb chondrocytes and cartilage (FIG. 8). Transcripts of components for ECM were generally more abundant from in utero than post-in utero lower limb chondrocytes, while those for secretory molecules showed more variable trends. Although the expression of genes encoding for type 2/9 collagen (COL2A1, COL9A1) was lower in week 15 MTOs, the expression from week 8 MTOs and week 11 MTOs fell in the range defined by examined human tissues. The expression of other major collagen components making up the non-hypertrophic region of cartilage (COL6A2 and COL/1.A ) and hypertrophic markers (COL10A1, MMP13) were not noticeably different between MTO and human lower limb cartilaginous tissues Among MTOs collected at different time points, despite falling in expression ranges of human tissues, week 15 MTOs showed slightly lower collagen contents and higher hypertrophic gene expression. As for transcripts of secretory molecules, week 15 MTOs showed similar expression (PGR4, CD44) to human tissues except for lower ACAN. The normal expression of PRG4, a gene encoding for a large proteoglycan synthesized by chondrocytes located at the surface of articular cartilage and by some synovial lining cells, offers additional support to the articular developmental trajectory spontaneously taken by MTO chondrocytes. In short, transcript levels of genes specifically expressed in human growth plate chondrocytes were strongly correlated between week 15 MTOs and human lower limb cartilaginous tissues.

Chondrogenesis and hyaline cartilage production in vitro is needed to provide chondrocytes for cartilage regenerative therapies as well as for ex vivo osteoarthritis modeling. Despite recent advances in hiPSC-based in vitro chondrogenesis, the development of simple and scalable protocols to generate chondrocytes for therapeutic purposes as well as the understanding of dynamic cell behaviors in long-term cultures are yet to be accomplished.

### IPSC-Derived MTOs for Osteoarthritis and Degenerative Cartilage Conditions

This disclosure describes the spontaneous emergence and robust growth of hyaline cartilage in hiPSC-derived MTOs grown in xeno-free and feeder-free cultures. The cultures do not require adding any growth factors or differentiation factors beyond what is present in E8 culture medium, making this protocol amenable for clinical good manufacturing practices (cGMP). Furthermore, the relative technical simplicity of the process also makes it suitable for robotic cell culture and scaled-up manufacturing. By characterizing and analyzing the transcriptome changes during the phenotypic transition of MTOs, we provide a mechanistic foundation for future organoid and related tissue engineering aiming at rapid cartilage production to thrive upon.

While described herein in the context of an exemplary embodiment in which the iPSCs used to generate the MTOs are human iPSCs (hiPSCs), the methods described herein may involve the use of iPSCs from other species when, for example, it is desired that the MTOs produce chondrogenic materials from a non-human species.

Generally, the method described herein involves culturing iPSCs in a culture medium that includes the fluid of hydration from a hydrogel-based 3D cell culture matrix rather than using the 3D hydrogel matrix itself. Culturing the iPSCs in medium that includes the fluid of hydration from is sufficient to induce the iPSCs to form organoids. Generally, the fluid of hydration for the hydrogel-based 3D cell culture matrix is a hyaluronic acid solution and with hyaluronic acid in a range of concentrations from 0.1% to 1.5%. However, in one or more embodiments, the iPSCs may be cultured in a physiologic solution (e.g., normal saline, phosphate buffered saline, Hank's balanced salt solution, DMEM, E8), either with or without hyaluronic acid.

Without wishing to be bound by any particular theory, iPSCs may generate MTOs in culture due to factors including, but not limited to, the absence of a 3D matrix and/or cell signaling that occurs between cultured iPSCs due to the cell density of the culture. These factors, rather than any particular culture medium components, may drive the generation of MTOs.

Cartilage in the organoids produced by the methods described herein resemble the hyaline cartilage typical of cartilage that exists during fetal development. Further, the hyaline cartilage produced by MTOs is similar in many respects to articular hyaline cartilage, including collagen type II, collagen type IX and aggrecan content. For example, higher aggrecan content in the ECM allows more capacity for the cartilage to withstand compression. Thus, the cartilage produced by the MTOs has biomechanical properties similar to articular cartilage, including, but not limited to, resistance to repetitive forces of compression and shear. Accordingly, the cartilage produced in the MTOs is useful for treatment of conditions that involve the degeneration of articular cartilage including, but not limited to, cartilage tears, osteochondral defects, osteoarthritis, etc.

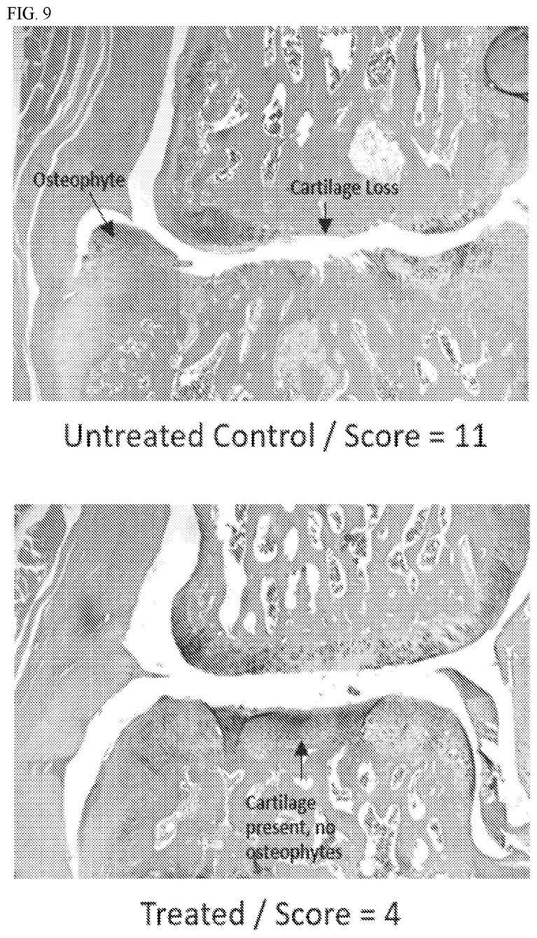

FIG. 9 shows histological data of treating osteoarthritis in a rat model. The top image (untreated control) and bottom image (treated with cells derived from chondrogenic organoids prepared without a 31) matrix) were scored according to the Osteoarthritis Research Society International (OARSI) method using a scale of 0-12, with 12 indicating severe osteoarthritis. The scoring regimen considers cartilage thickness, joint space thickness, presence of osteophytes, etc. Scoring was performed in a blinded manner. The top image, which received an OARSI score of 11, shows loss of cartilage and the presence of osteophytes. The bottom image, which received an OARSI score of 4, shows the presence of cartilage with only mild cartilage erosion and the absence of osteophytes. FIG. 10 shows immunohistochemistry staining of Ku80, indicating that human organoid-derived chondrocytes engraft to the treated knee in the rat model.

### Chondrospheres

Chondrospheres are structures formed from spontaneously from chondrocytes dissociated from MTOs. Chondrocytes may be obtained by physical dissociation of MTOs into single cells, then culturing in a bioreactor under conditions that the dissociated chondrocytes spontaneously form spheres of chondrocytes—i.e., chondrospheres—that have therapeutic activity. The dissociated cells may or may not be cultured on an ultra-low attachment surface so that the chondrocytes do not aggregate prior to being introduced into the bioreactor.

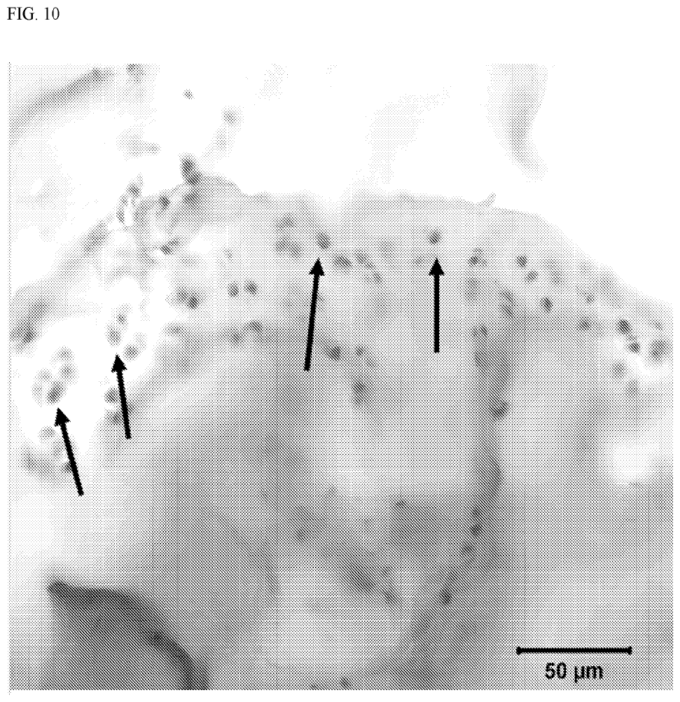

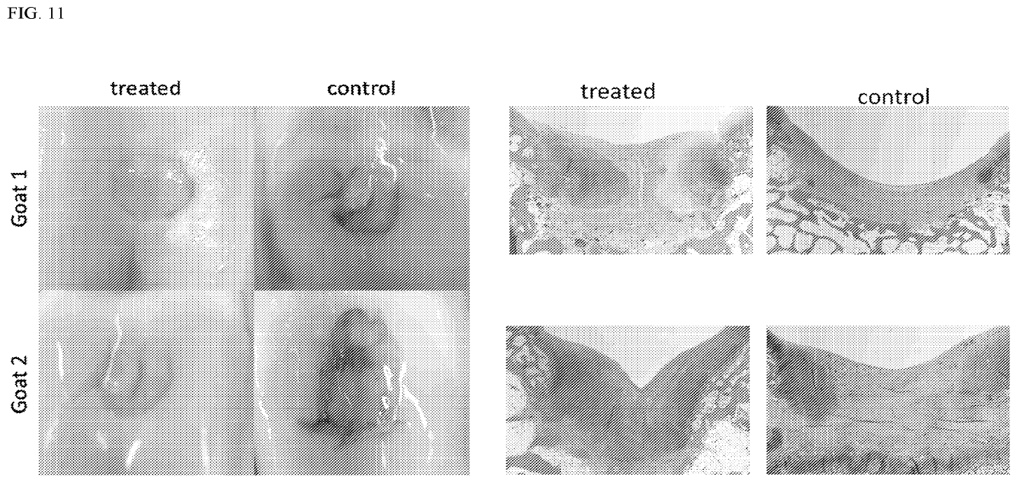

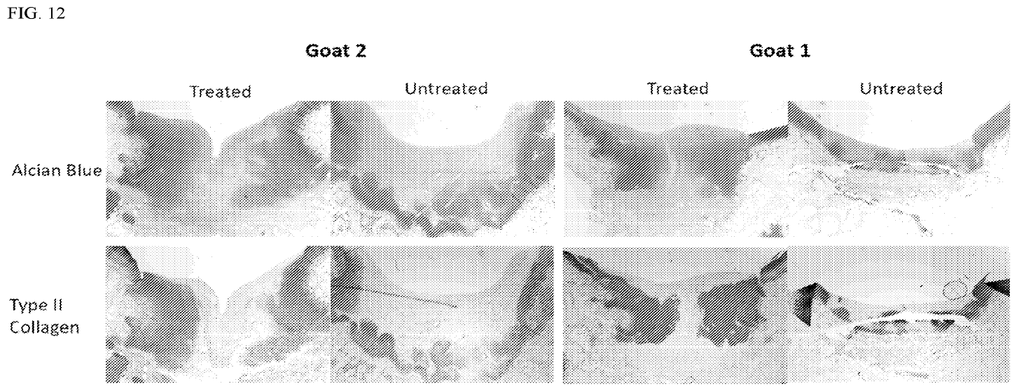

FIG. 11 and FIG. 12 show that human organoid-derived chondrospheres repair damaged cartilage in a goat model of surgically-induced articular cartilage injury in the goat femorotibial joint. In each goat an 8-mm diameter full-thickness defect was created in the articular cartilage of both femorotibial joints. In goat #1 (male), the treated side received single-cell chondrocyte product derived from MTO-derived chondrospheres embedded in fibrin glue (TISSEEL, Baxter International Inc., Deerfield, IL), and the control side received only fibrin glue. In goat #2 (female), the treated side received MTO-derived chondrospheres embedded in fibrin glue and the control side only received fibrin glue 12 weeks post-surgery, prominent articular cartilage regeneration is seen in the treated joint of both goat subjects as evidenced by the greater amount of Alcian blue staining tissue which demonstrates the proteoglycan/hyaluronic acid components typical of cartilage (FIG. 12). Immunohistochemistry of MTO cartilage also showed prominent amounts of the specific cartilage marker protein, type II collagen, in the regenerating articular cartilage in the treated joints of both subjects (FIG. 12).

### Chondrogenic Products and Methods

This disclosure therefore describes a scalable and cGMP-compatible method to generate authentic hyaline cartilage from chondrocytes and/or chondrospheres derived from organoids generated from human iPSCs. The chondrocytes maintain their chondrocyte phenotype, secrete collagen and anti-inflammatory factors, engraft into the damaged joint, and facilitate repair of cartilage damage in animal models (FIGS. 9-12). The methods described herein can be scaled to provide a high-capacity supply of therapeutic chondrocytes to treat conditions such as, but not limited to osteoarthritis. The organoid derived chondrocytes can thus overcome the disadvantages of the current cell-based procedures by providing an off-the-shelf chondrocyte therapy that does not require surgery to harvest patient cells or primary cell expansion. Further, the methods described herein produce uniform, high-quality chondrocytes that are not fibroblastic. Finally, the methods described herein use chemically defined materials and are readily adaptable to cGMP production and robotic scale-up.

iPSCs are an easily accessible, reproducible source of pluripotent cells, which can differentiate into chondrocytes that generate pure cartilage both in vitro and in vivo. Initial studies show no immune rejection of organoid-derived chondrocytes. Previous approaches for generating iPSC-derived chondrocytes have received limited study for therapeutic application in osteoarthritis because of the complexity and high cost of their generation and the significant variability associated with xenobiotic agents used in these manufacturing processes. To eliminate these shortcomings, the approach described herein generates chondrocytes in bulk from organoids generated from iPSCs without the use of xenobiotic substances or autologous donor cells.

Therapeutic Potential of iPSC Derived Human Chondrocytes for Musculoskeletal Conditions

Intra-articular injection of human chondrocytes is a logical therapeutic approach to treating osteoarthritis, but it has not been practical owing to the lack of readily available chondrocytes. Other cell-based approaches using mesenchymal stem/stromal cells may provide short term benefit, but they have not resulted in engraftment and long-term repair. Chondrocytes have potent anti-inflammatory capabilities that may reduce joint inflammation. Chondrocytes may engraft at sites of damaged cartilage and provide long-term repair and clinical improvement that may delay or obviate the need for total knee or hip replacement. Chondrocytes derived from organoids generated from iPSCs closely resemble juvenile chondrocytes, which are resistant to the inflammatory milieu of the osteoarthritic joint and have better proliferative capacity than primary adult chondrocytes. Juvenile chondrocytes therefore show enhanced survival and engraftment, enabling long-term efficacy. Importantly, allotransplantation of cartilage is known to be well-tolerated, supporting the potential for off-the-shelf use. The organoid-derived chondrocytes described herein have all the desirable qualities of juvenile chondrocytes, highlighting their extraordinary potential as a therapeutic for orthopedic medicine and surgery.

### Methods for Generating Organoids to Produce Cartilage

Methods for generating and using organoids to produce cartilage that involve culturing iPSCs in a 3D hydrogel matrix are described in, for example, International Publication Number WO 2020/132055 A1 and Lindborg et al., Stem Cells Transl Med 5(7):970-979 (2016). In those methods, culturing the cells in the 3D hydrogel matrix promotes growth of multi-tissue organoids (MTOs) from iPSCs. Those organoids contained primarily cerebral tissue, but could be manipulated to produce greater amounts of hyaline cartilage by controlling inductive and repressive signals for mesoderm specification in embryonic development.

In contrast, the methods described herein involve the spontaneous growth of hyaline cartilage in human iPSC-derived multi-tissue organoids (MTOs) without the use of a 3D hydrogel matrix and without having to manipulate culture conditions to redirect developmental pathways toward mesoderm specification. Rather, hyaline cartilage forms spontaneously using nothing more than standard cell culture medium (e.g., E8 medium), even in the absence of a 3D hydrogel cell culture matrix. The methods described herein are less laborious and more easily scaled up for clinical applications since the culture conditions do not require the use of a 3D hydrogel matrix or the use of serum-based factors to direct development of mesoderm tissues. Accordingly, multi-tissue organoids (MTOs) can be grown in a bioreactor and the hyaline cartilage contained in the MTOs can be isolated. The isolated hyaline cartilage may be dis-aggregated into individualized cells (e.g., chondrocytes), which can then be used to form a chondrocyte aggregate or can be cultured (e.g., on an ultra-low attachment surface) in chondrogenic media.

Characterization of Organoids that Produce Cartilage

Cartilage in the MTOs was identified histologically and by analysis of the transcriptome. The organoids grown in vitro using the methods described herein include chondrocytes with gene expression profiles similar to that of human fetal lower limb tissues. For example, RNA-seq data shows that the organoids contain chondrocytes having increases in expression of certain genes eight weeks, 11 weeks, and/or 15 weeks following MTO induction. Exemplary genes showing increased expression included, but were not limited to, bone morphogenic proteins (BMPs) and their intracellular signaling transducers (SMADs), and differentiation factors that induce chondrogenesis. Thus, exemplary genes whose expression is increased include, but are not limited to, transforming growth factor β1 (TGFβ1), fibroblast growth factor 2 (FGF2), bone morphogenic protein 2 (BMP2), bone morphogenic protein 6 (BMP6), growth differentiation factor 5 (GDF5), secreted frizzled related protein 1 (SFRP1), inhibin subunit βA (INHβA), transforming growth factor β3 (TGFβ3), insulin-like growth factor 2 (IGF2), leukemia inhibitory factor (LIF), bone morphogenic protein 4 (BMP4), BMP endothelial cell precursor-derived regulator (BMPER), left-right determination factor 1 (LEFTY1).

In another aspect, this disclosure describes an organoid, a cell of the organoid, a tissue of the organoid, a chondrocyte derived from the organoid, or a chondrocyte aggregate derived from the organoid generated using any embodiment of the methods described herein.

In another aspect, this disclosure describes cartilage generated using a multi-tissue organoid grown using any embodiment of the methods described herein. In some cases, the cartilage can be hyaline cartilage or articular cartilage.

In yet another aspect, the transcriptome analysis described herein indicate that multi-tissue organoids (MTOs) may serve as human-specific models for disease modeling and drug testing due to the developmental dynamics of chondrogenic pathways.

In summary, one aspect of this disclosure describes the long-term culture of iPSC-derived MTOs, resulting in the spontaneous emergence of mesoderm-derived articular cartilaginous tissues that resemble fetal limb bud and growth plate chondrocytes. The process described herein is self-organized with a comparably simpler, xenobiotic-free and feeder-free culturing protocol, making production easily adaptable to cGMP production and amenable to scaled-up commercial manufacturing.

### Neural MTO Products, Methods, and Potential Therapeutic Benefits

In another aspect, this disclosure describes a human midbrain organoid (MBO)-derived cell therapy to treat neurological disorders such as, for example, Parkinson's Disease. Parkinson's disease is a chronic progressive neurological disorder that most prominently affects movement. Currently available treatments for Parkinson's Disease (drugs and deep brain stimulation) attempt to compensate for the loss of dopamine by either providing the chemical precursor or by stimulating those brain regions that directly respond to the dopamine-producing midbrain cells. Unfortunately, these therapies are mainly palliative, eventually lose efficacy, may result in significant side effects, and/or do not prevent limit progression.

### Organoid Derived Cell-Based Therapies for Neurological Conditions

Organoids also can provide a source for cell therapeutics for neurological conditions. The use of midbrain organoids (MBOs) has several advantages over previously developed methods of neural cell production that recommend its use for commercial production. The method is technically simple, uses no animal-derived products, is scalable to produce large quantities of organoids at relatively low cost, does not require time-specific or dose-specific inclusion of growth factors, and is amenable to GMP production protocols.

Thus, in one aspect, this disclosure describes the use of organoids to generate cells that may be used for cell-based regenerative and/or restorative cell therapy for treating neurological disorders such as Parkinson's Disease. This therapy could restore the dopaminergic neurons and supporting cells lost in the course of this Parkinson's Disease, slow or stop further disease progression, and/or restore patient mobility. The cell therapeutic approach described herein can provide subject's having a neurological disorder a higher quality of life while reducing overall costs of care.

Human midbrain organoids (MBOs) involves inducing human iPS cells to form MBOs, which offers several advantages over previously developed methods of neural cell production that suggest the use of MBOs for commercial production of neuronal cells and tissues. The method is technically simple, uses no animal-derived products, is scalable to produce large quantities of organoids at relatively low cost, does not require time-specific and/or dose-specific inclusion of growth factors, and is amenable to GMP production protocols.

While described herein in the context of an exemplary embodiment in which the iPSCs used to generate the MTOs are human iPSCs (hiPSCs), the methods described herein may involve the use of iPSCs from other species when, for example, it is desired that the MTOs produce neurologic materials from a non-human species.

A regenerative and/or restorative cell therapy as described herein could restore dopaminergic neurons and supporting cells lost in the course of a neurodegenerative disease, slow or even stop further disease progression, and restore patient mobility. This cell therapeutic approach holds the promise to give patients with neurodegenerative disorders a higher quality of life while reducing overall costs of care.

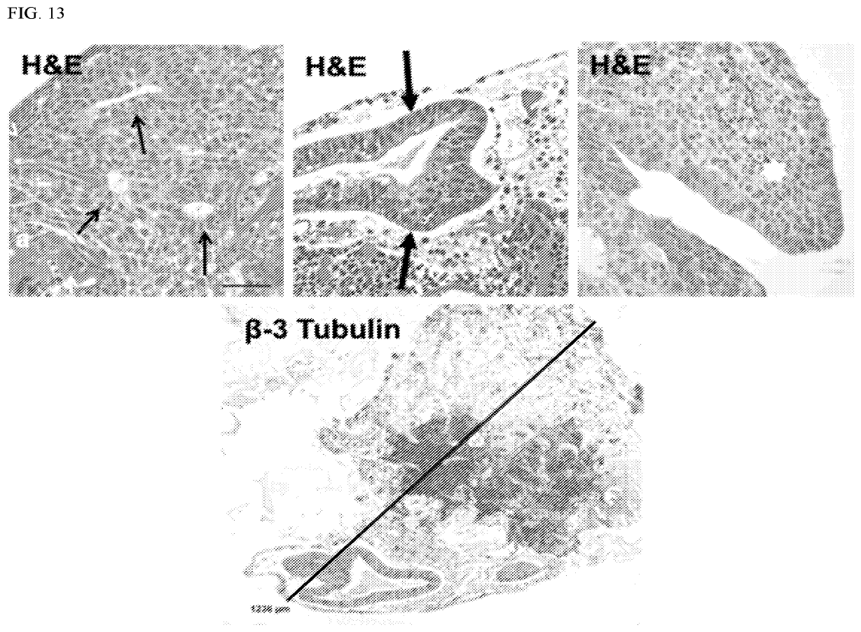

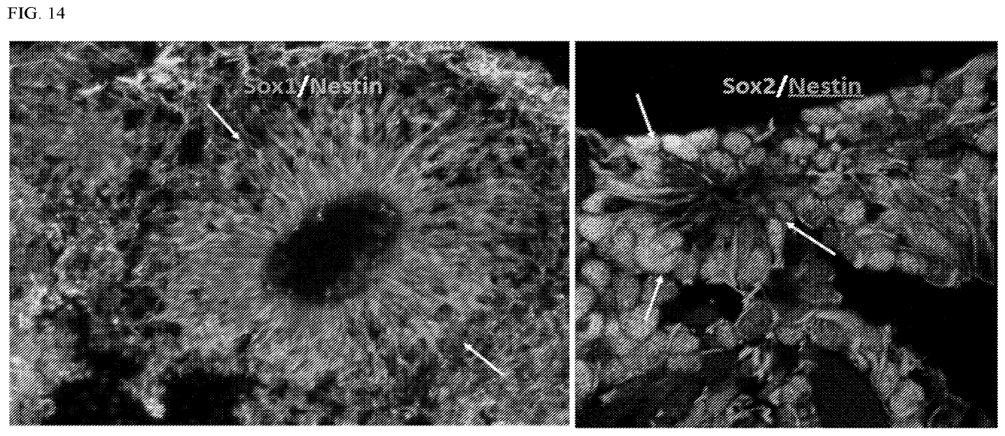

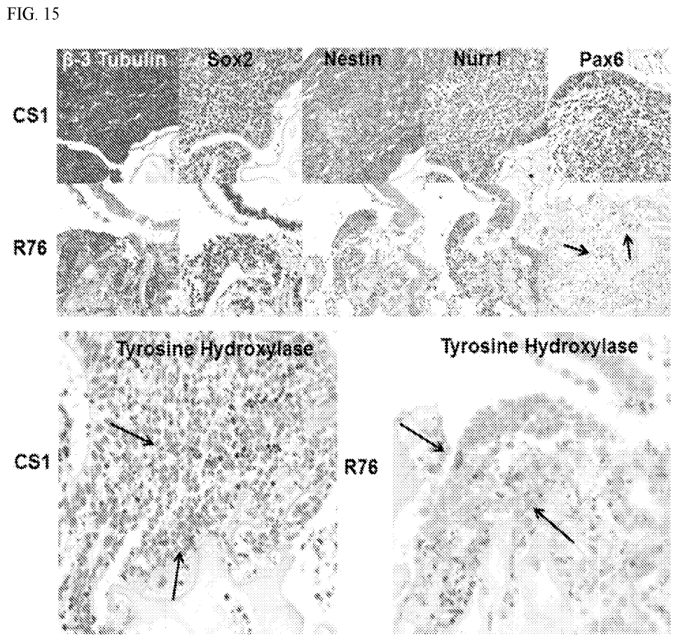

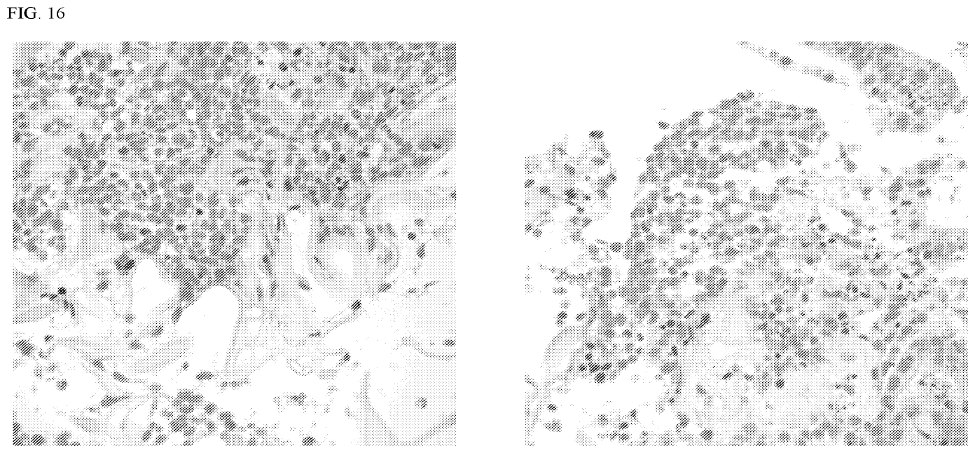

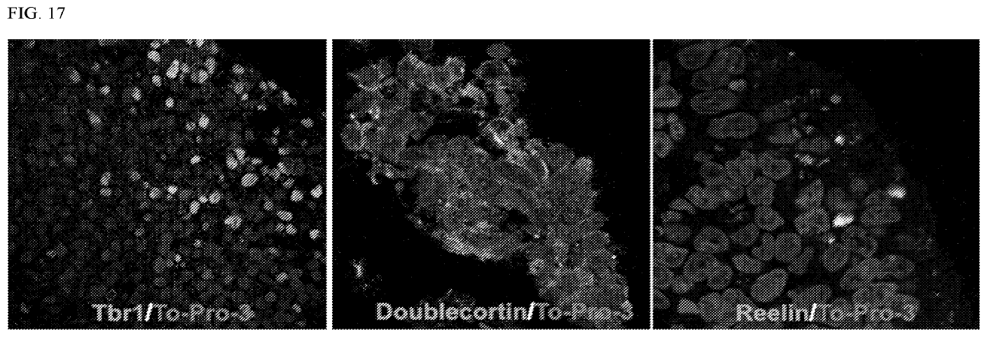

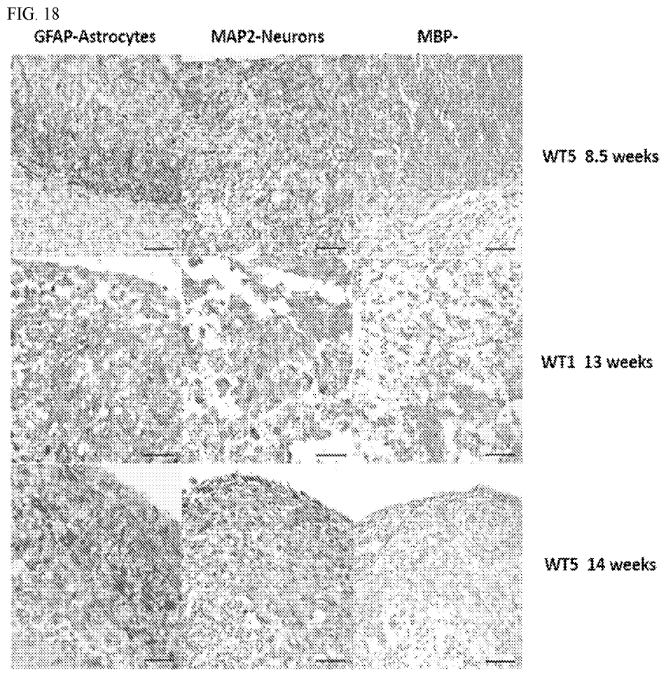

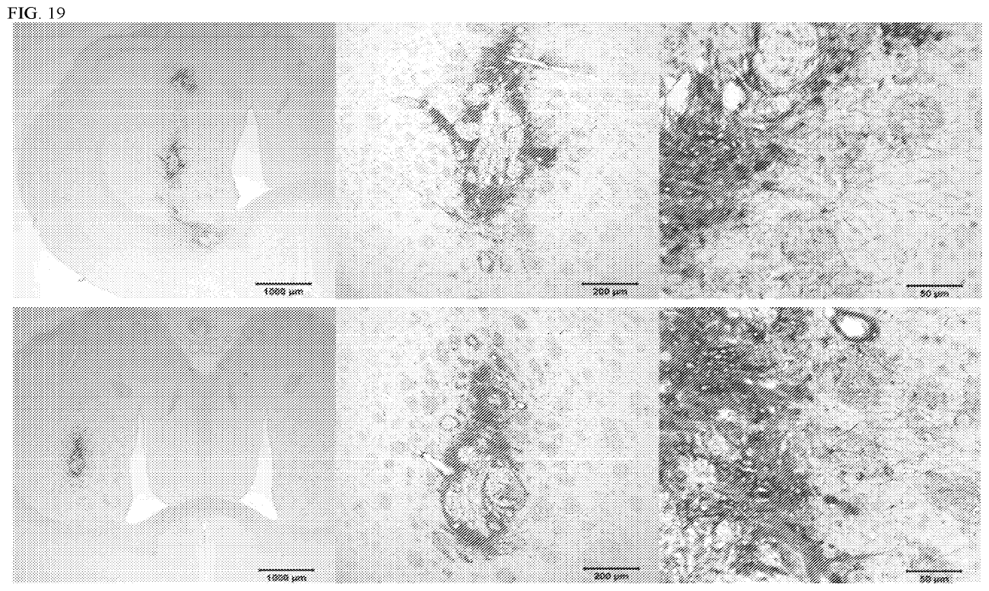

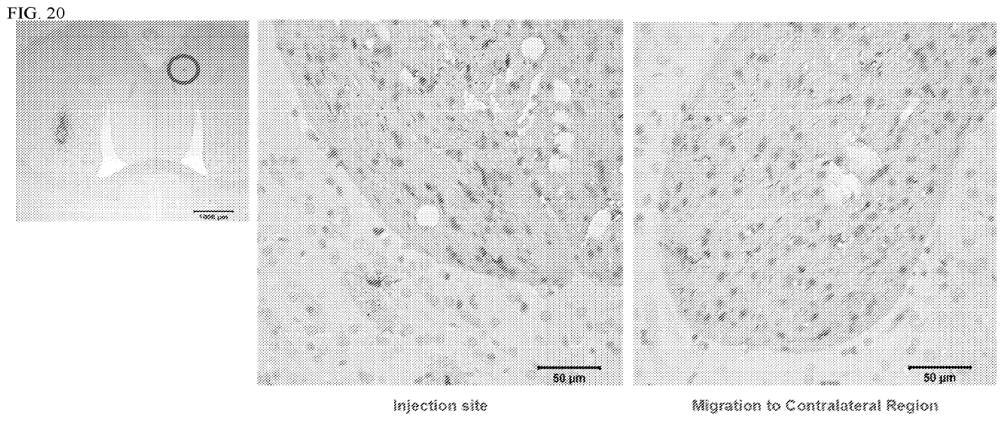

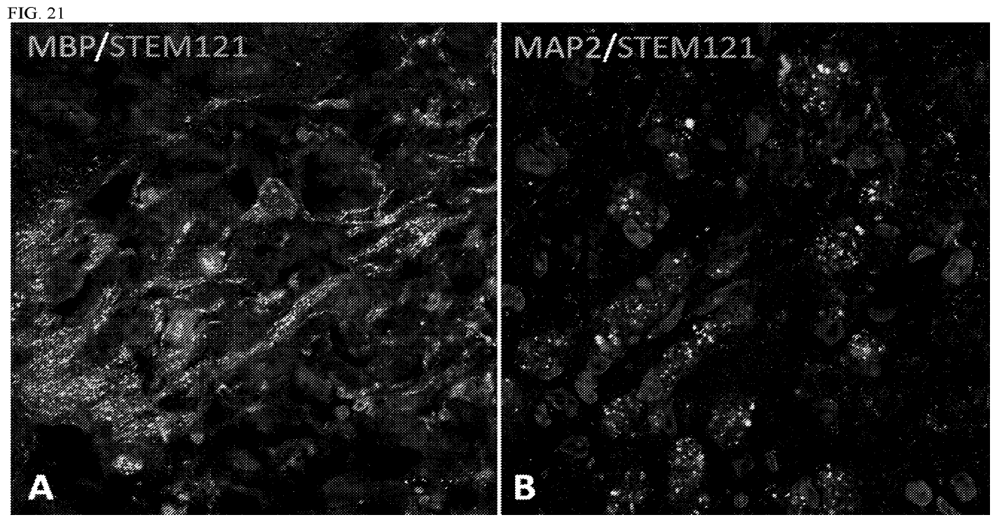

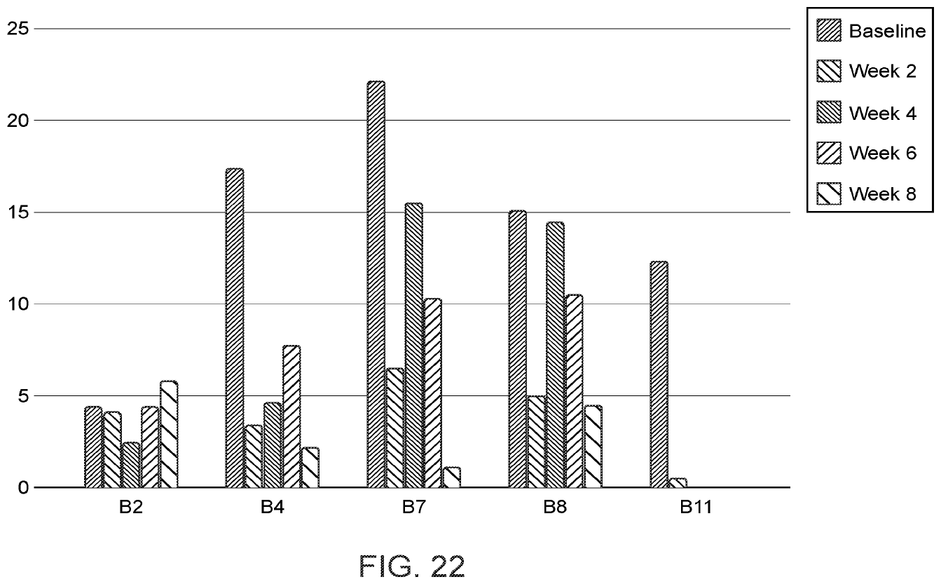

The brain organoid generating process described herein is robust and consistent. RNA-seq gene expression (2 lines) and immunohistochemical analysis of organoids (6 lines) showed multiple markers for mid-brain differentiation. Initial data suggest that MTO-derived cell product may have the potential to be a novel treatment of Parkinson's Disease. Pre-clinical data shows formation of neural rosettes, neural tube-like structures, and neural progenitors (cells double labeled for Sox1 or Sox2 and nestin) at early timepoints (FIG. 13; FIG. 14); the presence of dopaminergic neurons (tyrosine hydroxylase positive neurons) in MTOs derived from two different iPS cell lines (CS1 & R76, FIG. 15); the presence of oligodendrocyte progenitor cells (olig 2 positive cells, FIG. 16); markers of cerebral cortex formation (T-brain 1, doublecortin, and reelin, FIG. 17); the presence of mature astrocytes (glial fibrillary acidic protein, GFAP), neurons (microtubule-associated protein 2, MAP2), and oligodendrocytes (myelin basic protein, MBP) beyond week 6 (FIG. 18); engraftment resulting from the injection of 300,000 cells (immunohistochemical stain for STEM121 which is specific for human cells) derived from 6 week MTOs transplanted to the striatum region of rat brains (2 shown) with extension into surrounding brain and extensive migration to contralateral striatum 8 weeks post-transplantation (FIG. 19; FIG. 20) and with evidence of human oligodendrocyte (MBP/STEM121 double stain) and human neuron (MAP2/STEM121 double label) engraftment (FIG. 21); and a therapeutic benefit (e.g., reduced rotations) in rats (as in FIG. 19, FIG. 20, and FIG. 21) that had chemically-induced hemi-parkinsonism (rat model of Parkinson's Disease, FIG. 22).

### Compositions and Treatment Methods

Once isolated, MTO-derived materials (whether chondrogenic cells, chondrospheres, or neuronal cells) can be included in a pharmaceutical composition for administering to a subject. Thus, this disclosure describes pharmaceutical compositions that includes MTO-derived materials. This disclosure also describes methods of treating a subject having, or at risk of having, a condition treatable with the MTO-derived materials.

The subject can be a human or a non-human animal such as, for example, a livestock animal, a working animal, laboratory animal, or a companion animal. Exemplary non-human animal subjects include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, or reindeer), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

The MTO-derived materials described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the MTO-derived material, its use in the therapeutic compositions is contemplated. In one or more embodiments, the supplementary active ingredients also can be incorporated into the compositions. As used herein, “pharmaceutically acceptable” refers to a material that is not biologically or otherwise undesirable, i.e., the material may be administered to an individual along with MTO-derived material without causing any undesirable biological effects or interacting in a deleterious manner with any of the other components of the pharmaceutical composition in which it is contained.

The MTO-derived material may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, a pharmaceutical composition that includes MTO-derived material may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including, but not limited to, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the MTO-derived material into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of MTO-derived material administered can vary depending on various factors including, but not limited to, the weight, physical condition, and/or age of the subject, and/or the route of administration. Thus, the absolute number of MTO-derived material included in a given unit dosage form can vary widely, and depends upon factors such as the species, age, weight, and physical condition of the subject, and/or the method of administration. Accordingly, it is not practical to set forth generally the amount that constitutes an amount of MTO-derived material effective for all possible applications. Those of ordinary skill in the art, however, can readily determine the appropriate amount with due consideration of such factors.

In one or more embodiments, the method can include administering sufficient MTO-derived material to provide a dose of, for example, from about 10,000 cells to about 100 trillion cells to the subject, although in one or more embodiments the methods may be performed by administering MTO-derived material in a dose outside this range. In the context of chondrospheres, the term number of “cells” in a dose refers to the number of individual chondrocytes that form chondrospheres rather than the number of chondrospheres.

In one or more embodiments, the method can include administering sufficient MTO-derived material to provide a minimum dose of, for example, at least 10,000 cells, at least 50,000 cells, at least 100,000 cells, at least 200,000 cells, at least 300,000 cells, at least 400,000 cells, at least 500,000 cells, at least one million cells, at least two million cells, at least 10 million cells, at least 20 million cells, at least 50 million cells, at least 100 million cells, at least 500 million cells, at least one billion cells, at least five billion cells, at least 10 billion cells, at least 50 billion cells, or at least 100 billion cells.

In one or more embodiments, the method can include administering sufficient MTO-derived material to provide a maximum dose of, for example, no more than 100 trillion cells, no more than 50 trillion cells, no more than 10 trillion cells, no more than one trillion cells, no more than 500 billion cells, no more than 100 billion cells, no more than 50 billion cells, no more than billion cells, no more than 1 billion cells, or no more than 500,000 cells. Cells are said to be present in amounts “no more than” a reference amount when cells are not absent but are present in an amount up to the reference amount.

In one or more embodiments, the method can include administering sufficient MTO-derived material to provide a dose characterized by a range having endpoints defined by any a minimum dose identified above and any maximum dose identified above that is greater than the selected minimum dose. Thus, in one or more embodiments, the method can include administering sufficient MTO-derived material to provide a dose of, for example, 100,000 cells to 500,000, from 300,000 cells to one billion cells, from one billion cells to one trillion cells, from one million cells to 10 trillion cells, from 100 million cells to 500 billion cells, from 50 billion cells to 10 trillion cells, etc.

In certain embodiments, the method can include administering sufficient MTO-derived material to provide a dose equal to any minimum dose or any maximum dose listed above. Thus, for example, the method can include administering sufficient MTO-derived material to provide a dose of 300,000 cells, 500,000 cells, one million cells, one billion cells, 10 billion cells, 50 billion cells, 100 billion cells, one trillion cells, etc.

A single dose may be administered all at once, continuously for a prescribed period of time, or in multiple discrete administrations. When multiple administrations are used, the amount of each administration may be the same or different. For example, a dose of 100 billion cells per day may be administered as a single administration of 100 billion cells, continuously over 24 hours, as two or more equal administrations (e.g., two administrations of 50 billion cells), or as two or more unequal administrations (e.g., a first administration of 75 billion cells followed by a second administration of 25 billion cells). When multiple administrations are used to deliver a single dose, the interval between administrations may be the same or different.

In one or more embodiments, MTO-derived material may be administered, for example, from a single dose to multiple doses per week, although in one or more embodiments the method can involve a course of treatment that includes administering doses of the MTO-derived material at a frequency outside this range. When a course of treatment involves administering multiple doses within a certain period, the amount of each dose may be the same or different. For example, a course of treatment can include a loading dose initial dose, followed by a maintenance dose that is lower than the loading dose. Also, when multiple doses are used within a certain period, the interval between doses may be the same or be different.

In one or more embodiments, MTO-derived material may be administered from a once per week to a single once-off dose, although in one or more embodiments the methods may be performed by administering MTO-derived material at a frequency outside of this range.

Thus, in one or more embodiments, MTO-derived material may be administered at a minimum frequency of at least once per week, at least once per month, at least once per year, at least once every two years, at least once every three years, at least once five years, at least once every 10 years, or as a single once-off dose.

In one or more embodiments, MTO-derived material may be administered at a maximum frequency of no more than once every five years, no more than one every three years, no more than one every two years, no more than once per year, no more than one per month, or no more than once per week.

In one or more embodiments, MTO-derived material may be administered at a frequency characterized by a range having endpoints defined by any a minimum frequency identified above and any maximum frequency identified above that is more frequent than the selected minimum frequency. For example, in one or more embodiments, MTO-derived material may be administered at a frequency of from a once-off does to once per week, from once every three years to once per week, from once every five years to once per month, etc.

In certain embodiments, MTO-derived material may be administered at a frequency equal to any minimum frequency or any maximum frequency listed above. Thus, for example, MTO-derived material may be administered as a single once-off dose or at a frequency of once every three years, once every five years, etc.

In one or more embodiments, a course of treatment can have a duration of a single dose to the remaining life of the subject. Thus, in one or more embodiments, the course of treatment with MTO-derived material can have a minimum duration of a once-off dose, at least six months, at least one year, at least three years, at least five years, or until complete recovery.

In one or more embodiments, the course of treatment with MTO-derived material can have a maximum duration of the remaining life of the subject, no more than 10 years, no more than five years, no more than three years, no more than one year, no more than six months, or no more than three months.

In one or more embodiments, the duration of a course of treatment can be characterized by a range having endpoints defined by any a minimum duration identified above and any maximum duration identified above that is greater than the selected minimum duration. For example, in one or more embodiments, the duration of a course of treatment can be from a single once-off dose to the remaining life of the subject, from six months to five years, from three months to three years, etc.

In certain embodiments, the duration of the course of treatment can be equal to any minimum duration or any maximum duration listed above. Thus, for example, the duration of the course of treatment can be a single once-off dose, six months, three years, until complete recovery, or for the remaining life of the subject.

The pharmaceutical compositions described above can be used to treat a condition treatable using MTO-derived material. Treating a condition can be prophylactic or, alternatively, can be initiated after the subject exhibits one or more symptoms or clinical signs of the condition. As used herein, the term “symptom” refers to any subjective evidence of disease or of a patient's condition. As used herein, the term “sign” or “clinical sign” refers to an objective physical finding relating to a particular condition capable of being found by one other than the patient.

Treatment that is prophylactic—e.g., initiated before a subject manifests a symptom or clinical sign of the condition such as, for example, while an infection remains subclinical-is referred to herein as treatment of a subject that is “at risk” of having the condition. As used herein, the term “at risk” refers to a subject that may or may not actually possess the described risk. Thus, for example, a subject “at risk” of having a condition is a subject possessing one or more risk factors associated with the condition such as, for example, genetic predisposition, ancestry, age, sex, geographical location, lifestyle, or medical history. Treatment may also be continued after symptoms have resolved, for example to prevent or delay their recurrence.

Accordingly, a composition can be administered before, during, or after the subject first exhibits a symptom or clinical sign of the condition. Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

Thus, the method includes administering an effective amount of MTO-derived material to a subject having, or at risk of having, a condition treatable using MTO-derived material. In this aspect, an “effective amount” is an amount effective to reduce, limit progression, ameliorate, or resolve, to any extent, a symptom or clinical sign related to the condition.

### Conditions

The methods and MTO-derived products described herein can be used to treat any condition treatable using the MTO-derived materials. In one or more embodiments, the condition may be a condition treatable using chondrogenic MTO-derived material (e.g., chondrocytes and/or chondrospheres). Thus, the methods and MTO-derived material described herein may be effective for treating any condition that involves degeneration of articular cartilage. Accordingly, exemplary conditions include, but are not limited to, osteoarthritis of any joint (including, for example, the fingers, wrist, elbow, shoulder, hip, knee, ankle, toes, temporomandibular joint, etc.), cartilage injury, intervertebral disc disease, rheumatoid arthritis, hemochromatosis, psoriatic arthritis, gout, axial spondylarthritis, juvenile idiopathic arthritis, Saldino achondrogenesis, hypochondrogenesis, platyspondylic lethal skeletal dysplasia, Torrance type spondyloepiphyseal dysplasia congenita, Kniest dysplasia, SED with metatarsal shortening, Czech dysplasia, Spondyloperipheral dysplasia, Spondyloepimetaphyseal dysplasia (SEMD), Strudwick type, Stickler syndrome type 1, Mild SED with premature onset arthrosis, osteochondritis dissecans, relapsing polychondritis, chondrocalcinosis, osteochondroma, enchondroma, periosteal chondroma, multiple chondromatosis, enchondromatosis, chondroblastoma, chondromyxoid fibroma, systemic lupus erythematosus, seronegative spondyloarthropathies, temporomandibular arthritis, etc.

The chondrogenic MTO-derived material may be administered to a subject in an amount effective to, for example, promote regeneration of hyaline cartilage, promote production of Type II collagen, decrease the presence of osteophytes, decrease the severity and/or extent of a symptom or clinical sign of a disorder involving degeneration of articular cartilage (e.g., reduce inflammation, reduce pain, reduce swelling), or any combination of two or more of the foregoing.

In one or more embodiments, the condition may be a condition treatable using neuronal MTO-derived material. Thus, the methods and MTO-derived material described herein may be effective for treating ay condition that involves degeneration of neuronal cells or tissue. Accordingly, exemplary conditions include, but are not limited to, Parkinson's Disease, Alzheimer's disease, amyotrophic lateral sclerosis, Friedreich ataxia, Huntington's disease, Lewy body disease, spinal muscular atrophy, progressive supranuclear palsy, multiple system atrophy, or stroke.

The neuronal MTO-derived material may be administered to a subject in an amount effective to, for example, promote formation of neural rosettes in the subject, promote formation of neural progenitors in the subject, increase dopaminergic neurons in the subject, increase mature astrocytes in the subject, increase oligodendrocytes in the subject, increase markers of cerebral cortex formation in the subject, promote engraftment of the transplanted cells within brain tissue of the subject, increase migration of the transplanted cells within brain tissue of the subject, decrease the severity and/or extent of a symptom or clinical sign of a neurological disorder, or any two or more of the foregoing.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” or “one or more embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Generating hiPSC-Derived Multi-Tissue Organoids (MTOs)

Induced pluripotent stem cell (iPSC) lines referred to as 1024 (ATCC-BYS0110, Cat. #ACS-1024) and 9-1 (Lindborg et al., 2016, Stem Cells Transl Med 5(7):970-979; Ye et al., 2013, PLoS ONE 8(1):e53764) were expanded in culture on vitronectin (VTN-N, Thermo Fisher Scientific, Inc., Waltham, MA) in ESSENTIAL 8 medium (E8, Fujifilm Cellular Dynamics, Inc., Madison, WI). iPSCs were harvested using sodium citrate buffer, briefly centrifuged, and MTO induction initiated by resuspension in 40 μL of the fluid of hydration from Cell-Mate3D μGel 40 Kit (BRTI Life Sciences, Two Harbors, MN) which was then transferred to one well of a 6-well ultra-low attachment plate (COSTAR Ultra-Low Attachment Microplates, Corning Life Sciences, Corning, NY) containing 5 ml of E8 medium and incubated for 24 hours. Cells and culture medium were then transferred to a G-Rex 100 bioreactor (Wilson Wolf, New Brighton, MN) containing 25 ml of E8 medium and incubated at 37° C. in 5% C02. E8 culture medium containing 1% antibiotic-antimycotic (Gibco, Thermo Fisher Scientific Inc., Waltham, MA) was changed every 3-4 days over the entirety of MTO culture.

### Histology and Immunohistochemistry

MTOs from both cell lines were harvested at week 8 and week 11 (both cell lines) and week 30 (1024 only), placed in 10% neutral buffered formalin solution and fixed at room temperature for 3.5 hours. After fixation, samples were transferred to 70% ethanol solution until they were processed for routine paraffin embedding. Samples were then sectioned 4-mm thick, deparaffinized, rehydrated, and routinely stained with hematoxylin and eosin (H&E) and Alcian blue. For immunohistochemical staining, sections were cut at 4 mm, deparaffinized, and rehydrated, followed by incubation with 3% hydrogen peroxide to quench endogenous peroxidase activity and 15 minutes in serum-free protein block (DAKO, Glostrup, Denmark). Sections were then subjected to appropriate antigen retrieval methods (if needed) and incubated with the primary antibody at room temperature for 60 minutes. Color development was done using EnVision FLEX DAB+ substrate chromogen system (Cat. #GV825, Agilent-Dako, Santa Clara, CA). Stained sections were examined with an Olympus BH-2 microscope (Olympus America, Center Valley, PA) and imaged with a SPOT Insight 4 megasample digital camera and SPOT Advanced software (Diagnostic Instruments Inc., Sterling Heights, MI).

### Morphometry

Aggrecan immunohistochemistry-stained histologic sections of MTO biological replicates at 8 weeks (n=7) and 11 weeks (n=5) were analyzed for aggrecan staining area fraction (staining area/total area of tissue) using a Nikon Eclipse E-800M bright field/fluorescence/dark field microscope equipped with a Nikon DXM1200 high resolution digital camera. Images for histomorphometry were analyzed using ImageJ2/Fiji software (National Institutes of Health, open source). Values are reported as area fraction (%)±standard deviation.

### RNA-Seq of MTO and Data Generation.

MTOs were lysed in RLT buffer (Qiagen, Hilden, Germany) and RNA isolated from cell lysates using the RNeasy Plus mini kit (Qiagen, Hilden, Germany) according to manufacturer's instructions. Extracted RNA was then quantified by RiboGreen RNA assay (Thermo Fisher Scientific, Waltham, MA) and quality/size analyzed by Agilent BioAnalyzer (Agilent Technologies, Santa Clara, CA). 2×50 bp FastQ paired-end reads for six samples (n=62.4 million average per sample) were trimmed using Trimmomatic (v0.33) enabled with the optional “-q” option; 3 bp sliding-window trimming from 3′ end requiring minimum Q30. Quality control on raw sequence data for each sample was performed with FastQC. Read mapping was performed via Hisat2 (v2.1.0) using the Human genome (GRCh38) as reference. Gene quantification was done via Feature Counts for raw read counts. Existing RNA-seq data were processed using the same pipeline.

### RNA-Seq Data Analysis

Raw read counts (CPM) were used as input for differentially expressed genes (DGE) analysis by DESeq2 package (v1.30.1) in R (v4.0.5) (Love et al., Genome Biol. 15:550 (2014)). P-values were adjusted (p-adj) using Benjamini-Hochberg correction. Significant term was determined by using a cut-off of 0.05 (FDR corrected p<0.05) and minimum 2× Absolute Fold Change. TopGo package (v2.42.0; Alexa, A. and Rahnenfuhrer, J., topGO: Enrichment Analysis for Gene Ontology (2021)) was used to carry out ontology enrichment of DGE results, as previously described (Carbon et al., Nucleic Acids Res. 49:D325-D334 (2021)). Enrichment results were visualized using ClusterProfiler (v3.18.1; Yu et al., OMICS 16:284-287 (2012)). Cut-offs for p-value (after applying the Benjamini-Hochberg correction) and q-value were 0.05 and 1, respectively. Genes with expression FDR<0.05 and log 2 fold change greater or less than ±2 were selected and uploaded to STRING (v11) was used for functional protein association networks for probable gene products; Euclidean distances were used to cluster gene products, as previously described (Szklarczyk et al., Nucleic Acids Res 47:D607-D613 (2019)). Local network clusters were downloaded from STRING analysis, as previously described (Szklarczyk et al., Nucleic Acids Res 47:D607-D613 (2019)).

TABLE 3
MTO Gene Expression by RNA-seq at 11 and 15 weeks:
Assessment of Anti-Inflammatory/Regenerative
Factor Genes Known to be Secreted by MSCs

| | Week 11 | Week 15 |
|---|---|---|
| CXCL12 | 777.5 | 1337 |
| EGF | 227 | 187 |
| FGF7 | 59.5 | 1460 |
| FGF2 | 947 | 1909 |
| VEGFA | 4530 | 22479 |
| VEGFB | 7862 | 6536 |
| VEGFC | 148 | 1166 |
| PDGFA | 1596.5 | 3292 |
| PDGFB | 115 | 224 |
| PDGFC | 1986 | 4899 |
| HGF | 96 | 1661 |
| TGFB1 | 2546 | 5578 |
| IGF1 | 6 | 11 |
| IL1RN | 19.5 | 57 |
| IL6 | 4 | 206 |
| IDO1 | 51 | 28 |
| CD274 | 124 | 874 |
| LGALS1 | 10854 | 46501 |
| PTGES2 | 2057 | 1410 |
| GSTM2 | 1467 | 1108.5 |

### Additional Statistical Information

Unpaired t-test was used to compare histomorphometric measurements on MTO histologic sections. P-value was reported (α=0.05). Two biological replicates for MTO were used for RNA-Seq. All gene expression data used for visualization and statistical tests were first normalized and transformed using r log ( ) and assay( ) in DESeq2, as previously described (Love et al., Genome Biol. 15:550 (2014)). The one-tailed Wilcoxon signed-rank test was used to acquire statistical results for the change in expression of grouped genes; p-values were reported (α=0.05). The one-tailed pairwise t-test was used to compare the expression of single genes; p-values were reported (α=0.05). Confidence intervals were reported when applicable.

### Example 2

Human iPSCs were induced to form organoids as described in Example 1 but a buffered hyaluronic acid (HA) solution was used instead of the fluid of hydration from Cell-Mate3D μGel 40 Kit (BRTI Life Sciences, Two Harbors, MN). After culture in the low attachment plate, the iPSCs were transferred to E8 medium in the G-Rex 100 bioreactor flasks (Wilson Wolf, New Brighton, MN). The resulting hyaluronic acid-generated MTOs were then maintained in long-term cell culture for 12 weeks or 30 weeks in E8 medium in G-REX 100 bioreactor flasks (GREX, Wilson Wolf Inc., New Brighton, MN).

Cartilage/chondrocytes were present in the resulting multi-tissue organoids (MTOs). Developing hyaline cartilage was apparent after 12 weeks in the hyaluronic acid-generated MTO. After 30 weeks, mature hyaline cartilage morphology with chondrocytes surrounded by abundant matrix was observed and immunohistochemical (IHC) staining for aggrecan and type II collagen was consistent with hyaline cartilage.

### Example 3

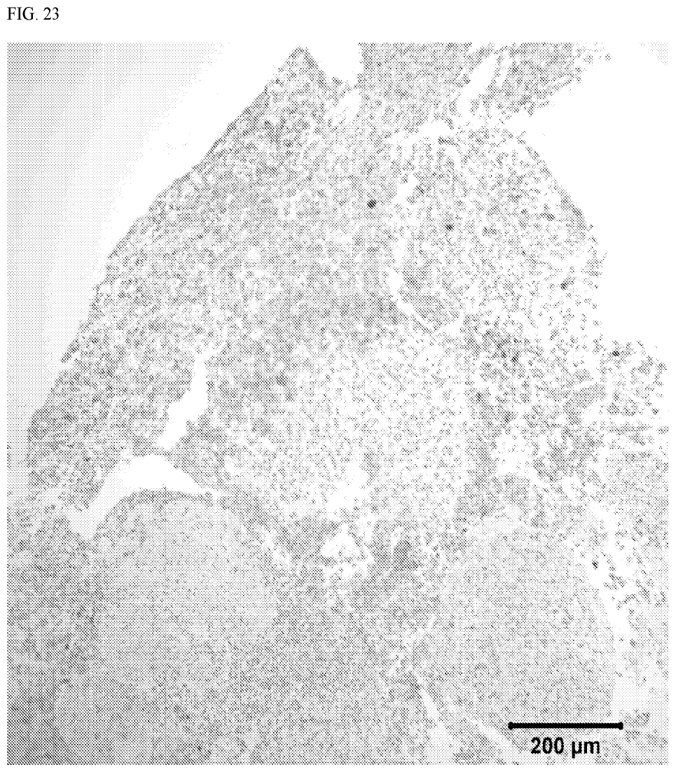

Same as Example 2, except that the input iPSCs were resuspended in a buffered hyaluronic acid solution and transferred directly into the G-REX 100 bioreactor for culture. This method yields neural and chondrogenic tissue. FIG. 23.

### Example 4

### Rat Model of Osteoarthritis

The treated joints received 2M chondrosphere-derived cells (SARCart cells, Sarcio, Inc., Minneapolis, MN) in Hank's Balanced Salt solution (HBSS) at surgery and again three weeks post-surgery. Control joints received HBSS only. Osteoarthritis lesions were scored from 0 (normal) to 12 (severe) by a pathologist blinded to treatment. FIG. 9 shows representative hematoxylin and eosin (H&E) staining of treated and untreated knee joints in one of six animals. The untreated knee showed moderate to severe osteoarthritis (scores 5-12) in the untreated control joints, and no disease to mild osteoarthritis (scores 0-4) in the treated joints, indicating a marked therapeutic effect of the organoid-derived chondrocytes. FIG. 10 Antibodies specific for human Ku80 (arrows) confirm engraftment of human SARCart cells in the treated rat osteoarthritis model. Control joints showed no Ku80 staining.

### Example 5

Goat model of Cartilage Injury Repair

FIG. 12 and FIG. 13. A cartilage injury was surgically induced by full thickness injuries, 8-mm diameter, in a non-weight-bearing area of articular cartilage of stifle (knee) joints in two goats. At surgery, lesions were treated as follows. In goat #1 (male), the treated side received single-cell chondrocyte product derived from MTO-derived chondrospheres which were embedded in fibrin glue (TISSEEL, Baxter International Inc., Deerfield, IL), and the control side received only fibrin glue. In goat #2 (female), the treated side received MTO-derived chondrospheres which were embedded in fibrin glue and the control side only received fibrin glue. 12 weeks after surgery the goats were sacrificed, and knees were examined by gross pathology before they were fixed in Neutral Buffered Formalin (NBF) and submitted for histopathologic examination Histology showed prominent neocartilage formation in treated lesions and minimal or no regeneration in control lesions.

### Example 6

### Generating Chondrospheres

Organoids were generated as described in EXAMPLE 1 or EXAMPLE 2. Organoids were harvested from the bioreactor, transferred to a 50-ml conical tube, and centrifuged at 200×g for five minutes. The supernatant was removed and the pellet was resuspended in 5 ml TRYPLE EXPRESS enzyme (Thermo Fisher Scientific, Inc., Waltham, MA) with 500 μg DNase 1 (Thermo Fisher Scientific, Inc., Waltham, MA). The organoids were incubated at 37° C. for five minutes, then mechanically dissociated with a 5-ml serological pipette. Dissociated cells were filtered using a 100-μm cell strainer (BD Falcon, Franklin Lakes, NJ) and organoids collected on the filter were transferred to a 50 ml conical tube containing 5 ml of 0.15% collagenase type II (Stem Cell Technologies, Inc., Vancouver, British Columbia, Canada) in DMEM/F12 with 500 μg of DNase 1 (Thermo Fisher Scientific, Inc., Waltham, MA).

The organoids were re-incubated at 37° C. for five minutes and subjected to a second round of mechanical dissociation using a 5-ml serological pipette. The mixture was filtered through a 100 μm cell strainer and the filter was washed with 5 ml DMEM/F12. Organoids collected on the filter were moved to a fresh a 50 ml conical tube containing 5 ml 0.15% collagenase (Stem Cell Technologies, Inc., Vancouver, British Columbia, Canada) in DMEM/F12 with 500 μg of DNase 1 (Thermo Fisher Scientific, Inc., Waltham, MA).

The organoids were re-incubated at 37° C. for five minutes and then subjected to mechanical dissociation using a p1000 pipette. The dissociated organoids were filtered through a 100-μm cell strainer and the filter was washed with 15 ml DMEM/F12 and the flow through was collected.

Cells from the flow through were counted, then centrifuged for five minutes at 200×g. The supernatant was removed. At this point, the organoids are fully dissociated into single cells and may be used as single cells or re-cultured in E8 medium to form chondrospheres.

To form chondrospheres, the dissociated organoid cell pellet was transferred to a G-Rex 100 bioreactor (Wilson Wolf, New Brighton, MN) with 35 ml E8 medium (Fujifilm Cellular Dynamics, Inc., Madison, WI) and incubated at 37° C. for approximately two weeks with media changed twice per week. Chondrospheres were collected in a 50-ml conical tube and centrifuged for five minutes at 200×g. The supernatant was removed and the pellet was suspended in 1 ml TRYPLE EXPRESS enzyme (Thermo Fisher Scientific, Inc., Waltham, MA) with 100 μg DNase 1 (Thermo Fisher Scientific, Inc., Waltham, MA), then incubated at 37° C. for five minutes.

The chondrospheres were manually dissociated five times with a p1000 pipette, then filtered through a 100-μm cell strainer. The cell strainer was then washed with at least 9 ml of DMEM/F12

If chondrospheres remained on the filter, they were collected and transferred to a fresh 50-ml conical tube containing 1 ml TRYPLE EXPRESS enzyme (Thermo Fisher Scientific, Inc., Waltham, MA) with 100 μg DNase 1 (Thermo Fisher Scientific, Inc., Waltham, MA), then incubated at 37° C. for five minutes. The chondrospheres were manually dissociated five times with a p1000 pipette, then filtered through a 100-μm cell strainer. The cell strainer was then washed with at least 9 mL of DMEM/F12.

Filtered cells were counted and incubated on ice. Cells were centrifuged at 200×g for five minutes. The supernatant was removed and the pellet was resuspended in E8 medium and distributed to multiple tubes in accordance to study parameters and intended dose per animal. For injection into animals, the chondrospheres were washed once in ice cold Hank's balanced salt solution (HBSS), centrifuged at 200×g for five minutes, then resuspended in HBSS. Rats received 2M cells suspended in 50 uL of HBSS via intra-articular injection. FIG. 9 and FIG. 10.

### Example 7

Neuronal MTO-derived material was generated by washing iPSCs three times with 17.5 ml PBS, then adding 17.5 ml passaging solution/citrate buffer into the flask containing the washed iPSCs. The cultures were observed for five minutes or until the cells began to lift off, at which point the passaging solution/citrate buffer was aspirated. The cells were then washed with 10 ml DMEM/F12 (Thermo Fisher Scientific, Inc., Waltham, MA) and collected into a 50 ml conical tube (total volume comes to 30 ml). The cells were centrifuged for five minutes at 150×g/1200 RPM. The supernatant was aspirated and the cell pellet was resuspended in 250 μl of CELL-MATE3D hydration fluid (BRTI Life Sciences, Two Harbors, MN) and vortexed according to manufacturer's protocol. The CELL-MATE3D matrix was transferred to a funnel apparatus and centrifuged to 2700 RPM according to the manufacturer's protocol.

A small piece (10 μl to 30 μl) of the CELLMATE3D matrix, containing cells, was cut using a scalpel and added to a G-REX 100 cell culture device (Wilson Wolf Corporation, St. Paul, MN) containing 50 mL ESSENTIAL 8 media (Thermo Fisher Scientific, Inc., Waltham, MA). The cells were incubated at 37° C. (5% CO₂, 20% O₂)) in the G-REX 100 cell culture device for 14-28 days, changing the culture medium every 3-4 days. Characteristics of resulting organoids are shown in FIG. 13-17

### Example 8

MTO derived neural tissue was prepared by embedding 20M iPSCs into a Cell-Mate3D micro gel per manufacturer's instructions and cultured in E8 medium. The cells were incubated at 37° C. (5% C02, 20% 02) in the G-REX 100 cell culture device (Wilson Wolf Corporation, St. Paul, MN) for 8.5-14 weeks (FIG. 18).

### Example 9

MTO-derived neural tissue prepared as described in EXAMPLE 8 was used in this example.

### Animals

Adult female nude rats (mu/mu; 210 g f 20 g; n=5) purchased from Taconic biosciences (Rensselaer, NY) for transplantation of 6-week organoid-derived cells for therapy of induced hemi-parkinsonism.

### 6-OHDA Induction of Hemi-Parkinsonism

Rats were anesthetized with vaporized isoflurane (Piramal Healthcare, Mumbai, India) and placed into a stereotaxic surgical frame (David Kopf Instruments, Inc., Tujunga, CA). The head of the rat was shaved and treated with iodine solution and ophthalmic gel applied to the eyes (VETERICYN PLUS, Innovacyn, Inc., Rialto, CA). A single midline incision was made along the scalp and skin retracted to expose bregma. A 10-μl Hamilton syringe (Hamilton Co., Reno, NV) was loaded with 3 μg/μl 6-hydroxydopamine hydrochloride (6-OHDA; Millipore-Sigma; St. Louis, MO) suspended in a solution of 1 mg/mL ascorbic acid in 0.9% NaCl (Hospira, Inc., Lake Forest, IL). A small burr hole was drilled in the skull above the injection site in the right hemisphere (from bregma: posterior 4.4 mm; lateral 1.2 mm). The needle was slowly inserted into the brain 7.6 mm ventral to the pia mater and 2.2 μl of the 6-OHDA solution was injected at a speed of 0.5 μl/minute. Following injection, the needle remained in place for two minutes before being slowly withdrawn. A second burr hole was then drilled in the skull above the second injection site in the right hemisphere (from bregma: posterior 4.0 mm; lateral 1.4 mm). The needle was slowly inserted into the brain 7.8 mm ventral to the pia mater and 1.8 μL of the 6-OHDA solution was injected at a speed of 0.5 μl/minute. At the conclusion of the injection the needle remained in place for two minutes before being slowly withdrawn. The incision site was cleaned and closed using a wound stapler (AUTOCLIP, Fine Science Tools, Foster City, CA). Rats were then placed in a heated recovery cage until fully sternal. Buprenorphine-SR (1 mg/kg; ZooPharm, Windsor, CO) was administered subcutaneously at the time of surgery.

### In Vivo Assessment of Therapeutic Response by Rotational Analysis

Ten days and 28 days following 6-OHDA lesion, and every two weeks following organoid transplantation, rats were tested for rotational bias. Rat were placed in clear plastic cylinders (38 cm diameter×34.5 cm height) underneath a ceiling mounted video camera. An initial 10-minute acclimation period was followed by a 20-minute recording when injected intraperitoneally with 0.9% NaCl. Rats were then injected intraperitoneally with 5 mg/kg D-amphetamine (Millipore-Sigma, Burlington, MA) in 0.9% NaCl and recorded for 40 minutes. Recording were analyzed using code written by the University of Minnesota Imaging Center staff using Fiji imaging software (Schindelin et al., 2012, Nature Methods 9:676-682). Automated counts were confirmed in a subset of animals through visual counts. The number of clockwise and anti-clockwise rotations were counted for each animal and calculated as average rotations per minute. Animals with an average D-amphetamine rotational score of seven clockwise rotations per minute were included in transplantation studies. All rotational scores following transplantation are reported as a percentage of rotations relative to baseline (28 days post-lesion).

### Preparation of Organoids for Transplantation

Prior to transplantation midbrain organoids were dissociated into a single cell suspension. Briefly, organoids were rinsed in PBS then treated with 2 ml 0.05% Trypsin-EDTA (Life Technologies, Inc., Carlsbad, CA) for two minutes at 37° C. An additional 2 ml Trypsin-EDTA supplemented with 200 μg DNase1 (Millipore-Sigma, Burlington, MA) was added and mechanically dissociated using a p1000 pipette. The organoids were then incubated for five minutes at 37° C. after which, cells were again mechanically dissociated and cold Hank's Balanced Salt solution was added to bring the final volume to 10 ml. The cells were centrifuged at 250×g for five minutes at 4° C. The resulting supernatant was removed and cell pellet resuspended in 10 ml of cold HBSS and passed through a 70-μm nylon cell strainer (BD Biosciences, San Jose, CA). The cells were centrifuged a second time and the resulting pellet was resuspended in 1 ml cold HBSS for counting using a hemocytometer. The cells were centrifuged a third time and resuspended at a concentration of roughly 5×10⁴ cells per μl of cold HBSS. The final cell solution was counted and viability was assessed using the Trypan Blue exclusion method. The final cell count was calculated as the total number of viable cells per μl.

### Transplantation

The head of the rat was shaved and treated with betadine. A single midline incision was made along the scalp and skin retracted to expose bregma. A 10-μl Hamilton syringe (Hamilton Co., Reno, NV) was loaded with the cell solution. A small burr hole was drilled in the skull above the injection site in the right hemisphere (from bregma: anterior 1.0 mm; lateral 3.0 mm). The needle was slowly inserted into the brain 6.5 mm ventral to the pia mater and 1×10⁵ viable cells were injected at a speed of 0.5 μl/minute. Following injection, the needle remained in place for one minute. The injection was repeated at 5.5 mm and 4.5 mm ventral to the pia mater with 1×10⁵ cells injected at each site for a total of 3×10⁵ viable cells. At the conclusion of the last injection the needle remained in place for three minutes before being slowly withdrawn. The incision site was cleaned and closed using a wound stapler (AUTOCLIP, Fine Science Tools, Foster City, CA).

### Tissue Collection

At eight weeks post-transplantation, rats were deeply anesthetized using vaporized isoflurane. Rats were transcardially perfused with ice-cold PBS followed by ice-cold 4% paraformaldehyde fixative. The brain was removed and placed in fixative overnight at 4° C. Fixed tissues were processed routinely for paraffin embedding, sectioned at 4 μm thick, deparaffinized, rehydrated, and stained with hematoxylin and eosin and immunohistochemically for the STEM121 antigen to demonstrate human cells.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. (canceled)

2. A method of producing a multi-tissue organoid (MTO), the method comprising:
introducing a pluripotent stem cell into a cell culture medium comprising hyaluronic acid;
transferring the pluripotent stem cell to a cell culture device that does not include a three-dimensional matrix;
culturing the pluripotent stem cell in the cell culture device for at least 1 week; and
producing a multi-tissue organoid comprising cartilage, bone, fibrous connective tissue, brain tissue, or epithelial tissue, or a combination thereof.

3. The method of claim 2, wherein the cell culture medium further comprises one or more of fibroblast growth factor, transforming growth factor beta (TGFβ), growth differentiation factor 5 (GDF-5), DMEM/F12, L-ascorbic acid-2-phosphate magnesium, sodium selenium, insulin, NaHCO₃, transferrin, TGFβ1, NODAL, or bone morphogenetic protein 2 (BMP-2).

4-6. (canceled)

7. The method of claim 2, wherein the PSCs are incubated in a bioreactor.

8. The method of claim 2, wherein the organoid comprises cartilage, bone, fibrous connective tissue, or a combination thereof.

9. The method of claim 8, wherein the cartilage comprises hyaline cartilage.

10. The method of claim 2, wherein the organoid comprises neuronal cells or neuronal tissue.

11. The method of claim 2, further comprising isolating the organoid.

12. The method of claim 2, further comprising: dis-aggregating the cells of the organoid to produce a population of individualized cells.

13. The method of claim 12, further comprising culturing a cell from the population of individualized cells.

14-20. (canceled)

21. The method of claim 2, wherein the cell culture device lacks a biomimetic coating.

22. A method of treating a subject having, or at risk of having a condition that includes degeneration of articular cartilage, the method comprising administering to the subject a composition that includes chondrogenic MTO-derived material in an amount effective to ameliorate at least one symptom or clinical sign of the condition.

23. The method of claim 22, wherein the MTO-derived material comprises a chondrocyte, a chondrosphere, or both.

24. The method of claim 22, wherein the chondrogenic MTO-derived material is administered in an amount effective to promote regeneration of hyaline cartilage, promote production of Type H collagen, decrease the presence of osteophytes, reduce joint pain, reduce joint inflammation, or any combination of two or more of the foregoing.

25. The method of claim 22, wherein the condition is osteoarthritis, cartilage injury, intervertebral disc disease, rheumatoid arthritis, hemochromatosis, psoriatic arthritis, gout, axial spondylarthritis, or juvenile arthritis, Saldino achondrogenesis, hypochondrogenesis, platyspondylic lethal skeletal dysplasia, Torrance type Spondyloepiphyseal dysplasia congenita, Kniest dysplasia, SED with metatarsal shortening, Czech dysplasia, Spondyloperipheral dysplasia, Spondyloepimetaphyseal dysplasia (SEMD), Strudwick type, Stickler syndrome type 1, Mild SED with premature onset arthrosis, Osteochondritis dissecans, Relapsing polychondritis, Chondrocalcinosis, osteochondroma, enchondroma, periosteal chondroma, multiple chondromatosis, enchondromatosis, chondroblastoma, chondromyxoid fibroma, rheumatoid arthritis, juvenile idiopathic arthritis, gout, systemic lupus erythematosus, seronegative spondyloarthropathies, or temporomandibular arthritis.

26. A method of treating a subject having, or at risk of having a condition that includes degeneration of degeneration of neuronal cells or neuronal tissue, the method comprising administering to the subject a composition that includes neuronal MTO-derived material in an amount effective to ameliorate at least one symptom or clinical sign of the condition.

27. The method of claim 26, wherein the neuronal MTO-derived material is administered in an amount effective to promote formation of neural rosettes in the subject, promote formation of neural progenitors in the subject, increase dopaminergic neurons in the subject, increase mature astrocytes in the subject, increase oligodendrocytes in the subject, increase markers of cerebral cortex formation in the subject, promote engraftment of the transplanted cells within brain tissue of the subject, increase migration of the transplanted cells within brain tissue of the subject, decrease the severity and/or extent of a symptom or clinical sign of a neurological disorder, or any two or more of the foregoing.

28. The method of claim 26, wherein the condition is Parkinson's Disease, Alzheimer's disease, amyotrophic lateral sclerosis, Friedreich ataxia, Huntington's disease, Lewy body disease, spinal muscular atrophy, progressive supranuclear palsy, multiple system atrophy, or stroke.

29. The method of claim 23, wherein the chondrogenic MTO-derived material is administered in an amount effective to promote regeneration of hyaline cartilage, promote production of Type II collagen, decrease the presence of osteophytes, reduce joint pain, reduce joint inflammation, or any combination of two or more of the foregoing.

30. The method of claim 23, wherein the condition is osteoarthritis, cartilage injury, intervertebral disc disease, rheumatoid arthritis, hemochromatosis, psoriatic arthritis, gout, axial spondylarthritis, or juvenile arthritis, Saldino achondrogenesis, hypochondrogenesis, platyspondylic lethal skeletal dysplasia, Torrance type Spondyloepiphyseal dysplasia congenita, Kniest dysplasia, SED with metatarsal shortening, Czech dysplasia, Spondyloperipheral dysplasia, Spondyloepimetaphyseal dysplasia (SEMD), Strudwick type, Stickler syndrome type 1, Mild SED with premature onset arthrosis, Osteochondritis dissecans, Relapsing polychondritis, Chondrocalcinosis, osteochondroma, enchondroma, periosteal chondroma, multiple chondromatosis, enchondromatosis, chondroblastoma, chondromyxoid fibroma, rheumatoid arthritis, juvenile idiopathic arthritis, gout, systemic lupus erythematosus, seronegative spondyloarthropathies, or temporomandibular arthritis.

31. The method of claim 24, wherein the condition is osteoarthritis, cartilage injury, intervertebral disc disease, rheumatoid arthritis, hemochromatosis, psoriatic arthritis, gout, axial spondylarthritis, or juvenile arthritis, Saldino achondrogenesis, hypochondrogenesis, platyspondylic lethal skeletal dysplasia, Torrance type Spondyloepiphyseal dysplasia congenita, Kniest dysplasia, SED with metatarsal shortening, Czech dysplasia, Spondyloperipheral dysplasia, Spondyloepimetaphyseal dysplasia (SEMD), Strudwick type, Stickler syndrome type 1, Mild SED with premature onset arthrosis, Osteochondritis dissecans, Relapsing polychondritis, Chondrocalcinosis, osteochondroma, enchondroma, periosteal chondroma, multiple chondromatosis, enchondromatosis, chondroblastoma, chondromyxoid fibroma, rheumatoid arthritis, juvenile idiopathic arthritis, gout, systemic lupus erythematosus, seronegative spondyloarthropathies, or temporomandibular arthritis.

32. The method of claim 27, wherein the condition is Parkinson's Disease, Alzheimer's disease, amyotrophic lateral sclerosis, Friedreich ataxia, Huntington's disease, Lewy body disease, spinal muscular atrophy, progressive supranuclear palsy, multiple system atrophy, or stroke.
